(19) United States
(12) Patent Application Publication
(10) Pub. No.: US 2022/0390357 A1
(43) Pub. Date: Dec. 8, 2022

(54) EVALUATION DEVICE, EVALUATION METHOD, AND PROGRAM

(71) Applicant: University of Tsukuba, Tsukuba-shi, Ibaraki (JP)

(72) Inventors: Yoshiaki Yasuno, Tsukuba-shi (JP); Pradipta Mukherjee, Tsukuba-shi (JP); Ibrahim Gamal Abdelsadek Hossein, Tsukuba-shi (JP); Arata Miyazawa, Tsukuba-shi (JP)

(21) Appl. No.: 17/776,191

(22) PCT Filed: Nov. 13, 2020
(86) PCT No.: PCT/JP2020/042497

§ 371 Date: May 11, 2022

(30) Foreign Application Priority Data
Nov. 15, 2019 (JP) 2019-207348
Apr. 9, 2020 (JP) 2020-070309

(51) Int. Cl. G01N 21/23; G01N 33/483

(52) U.S. Cl. CPC G01N 21/23; G01N 33/4833; G01N 2021/1787

(57) ABSTRACT

Provided is an evaluation method for a biological tissue that enables dynamics of the biological tissue to be quantitatively evaluated. In the evaluation method of the present embodiment, an optical coherence tomography (OCT) signal indicating a state of a biological tissue provided as a sample is acquired, a signal value based on the OCT signal is acquired at an observation point in the sample, and a temporal variation characteristic value indicating a temporal variation characteristic of the signal value within a predetermined period is calculated. The present embodiment can also be implemented with an evaluation device or even with a program.

### TECHNICAL FIELD

The present invention relates to an evaluation device and method, and a program. The invention relates to a technique for visualizing and quantitatively evaluating a state of a sample of a biological tissue or the like by processing a measurement signal obtained from measurement in, for example, optical coherence tomography (OCT). The present application claims priority to Japanese Patent Application No. 2019-207348 filed in Japan on Nov. 15, 2019, and Japanese Patent Application No. 2020-070309 filed in Japan on Apr. 9, 2020, the contents of which are incorporated herein by reference.

### BACKGROUND ART

In recent years, a technique called “OCT microscope” for imaging ex vivo samples using OCT has been studied. However, the OCT microscope is generally of a technique for morphological imaging, and is not capable of imaging tissue functions such as metabolism. Meanwhile, a signal analysis method called “dynamic OCT” has been proposed (Non-Patent Literature 1). However, this method has poor quantitativeness, making it difficult to correctly evaluate the degree of biological activities. In addition, this method is suitable for a special type of OCT called Full-field OCT (FF-OCT) and it is difficult to implement this method in widely used scanning OCT.

### CITATION LIST

### Non-Patent Literature

[Non-Patent Literature 1] Apelian et al., “Dynamic Full Field Optical Coherence Tomography: Subcellular Metabolic Contrast Revealed in Tissues by Interferometric Signals Temporal Analysis”, Biomedical Optics Express 7, No. 4, p. 1511-1524, Mar. 24, 2016

### SUMMARY OF INVENTION

### Technical Problem

The present invention has been conceived taking the above-described circumstances into account and one objective of the present invention is to provide an evaluation method that enables quantitative evaluation of dynamic characteristics of samples, for example, dynamics and intracellular activities of biological tissues.

### Solution to Problem

The present invention employs the following means to solve the above-described problems.

(1) An aspect of the present invention is an evaluation device including a measurement unit that acquires an optical coherence tomography (OCT) signal indicating a state of a biological tissue provided as a sample and acquires a signal value based on the OCT signal at an observation point in the sample and an evaluation unit that calculates a temporal variation characteristic value indicating a temporal variation characteristic of the signal value within a predetermined period.

(2) In another aspect of the present invention, the evaluation unit may calculate a variance of the signal value as the temporal variation characteristic value.

(3) In another aspect of the present invention, the evaluation unit may divide a sum of squares of a deviation between a signal intensity of the OCT signal and a mean value of the signal intensity at a frame time within the predetermined period by the number of frames in the predetermined period to calculate the variance at the observation point.

(4) In another aspect of the present invention, the evaluation unit may calculate a correlation coefficient of the signal value and a time-shifted signal value obtained by time-shifting the signal value by a time shift amount τ for each time shift amount τ, and calculate a decay speed of the correlation coefficient according to an increase in the time shift amount τ as the temporal variation characteristic value.

(5) In another aspect of the present invention, the evaluation unit may calculate, as a variance, a sum of squares of a deviation between a signal intensity of the OCT signal and a mean value of the signal intensity at a frame time within the predetermined period, calculate, as a covariance, a sum of a product of a deviation between a signal intensity of the OCT signal and a mean value of the signal intensity at a frame time within the predetermined period and another deviation between a time-shifted signal intensity of the OCT signal at a shift time shifted from the frame time by a time shift amount τ and a mean value of the time-shifted signal intensity, calculate the correlation coefficient by dividing the covariance by the variance for each shift amount τ, and perform regression analysis using a predetermined decay function using the correlation coefficient for each time shift amount τ and calculate a parameter of the decay function approximating the correlation coefficient, as the decay speed at an observation point.

(6) In another aspect of the present invention, the evaluation unit may calculate the decay speed using the correlation coefficient calculated with the time shift amount τ being non-zero.

(7) In another aspect of the present invention, the measurement unit may determine a polarization characteristic value based on a polarization characteristic at an observation point in the sample, based on a first measurement signal of a first interferometric component in a first polarization state, the first interferometric component being obtained by causing a first incidence component incident on the sample in the first polarization state to interfere with a component obtained by reflection or scattering of the first incidence component from the sample, a second measurement signal in a second polarization state with respect to the first interferometric component, a third measurement signal of a second interferometric component in the first polarization state, the second interferometric component being obtained by causing a second incidence component incident on the sample in the second polarization state to interfere with a component obtained by reflection or scattering of the second incidence component from the sample, and a fourth measurement signal in the second polarization state with respect to the second interferometric component, and the evaluation unit may determine the temporal variation characteristic value indicating a temporal variation characteristic of the polarization characteristic value.

(8) In another aspect of the present invention, the measurement unit may determine a Jones matrix at an observation point based on the first measurement signal, the second measurement signal, the third measurement signal, and the fourth measurement signal and determine a cumulative Jones matrix at the observation point from a Jones matrix at the observation point in the sample and a Jones matrix on a surface of the sample, and determine, as the polarization characteristic value, a cumulative phase retardation index value that is a phase difference between eigenvalues of the cumulative Jones matrix.

(9) In another aspect of the present invention, the measurement unit may determine a Jones matrix at an observation point based on the first measurement signal, the second measurement signal, the third measurement signal, and the fourth measurement signal and determine, from a Jones matrix at a first observation point in the sample and a Jones matrix at a second observation point in the sample, a local Jones matrix between the first observation point and the second observation point, and determine the polarization characteristic value based on a local phase retardation that is a phase difference between eigenvalues of the local Jones matrix.

(10) In another aspect of the present invention, the measurement unit may determine a birefringence by dividing the local phase retardation by a wavenumber of incident light incident on the sample and a thickness between the first observation point and the second observation point.

(11) In another aspect of the present invention, the evaluation unit may calculate the temporal variation characteristic value based on a variance or a standard deviation of the polarization characteristic value.

(12) In another aspect of the present invention, the evaluation unit may calculate the temporal variation characteristic value based on a variance or a standard deviation of a logarithmic value of the polarization characteristic value.

(13) In another aspect of the present invention, the evaluation unit may calculate a dynamic contrast by dividing the standard deviation of the polarization characteristic value by a mean value of the birefringence.

(14) In another aspect of the present invention, the measurement unit may convert, as the polarization characteristic values, a first Jones vector based on the first measurement signal and the second measurement signal and a second Jones vector based on the third measurement signal and the fourth measurement signal into a first Stokes vector and a second Stokes vector, respectively, and the evaluation unit may determine a temporal polarization uniformity based on a time average of the first Stokes vectors and a time average of the second Stokes vectors as the temporal variation characteristic value.

(15) In another aspect of the present invention, the measurement unit may determine a temporal polarization uniformity based on a time average of a corrected first Stokes vector obtained by subtracting a noise component from the first Stokes vector and a time average of a corrected second Stokes vector obtained by subtracting a noise component from the second Stokes vector.

(16) In another aspect of the present invention, the measurement unit may determine, as the polarization characteristic value, a Jones matrix at an observation point based on the first measurement signal, the second measurement signal, the third measurement signal, and the fourth measurement signal, and the evaluation unit may calculate a von Neumann entropy of the Jones matrix as the temporal variation characteristic value.

(17) In another aspect of the present invention, the evaluation unit may calculate an entropy of a noise component from a temporal polarization uniformity of a first Stokes vector and a temporal polarization uniformity of a second Stokes vector, the first Stokes vector and the second Stokes vector being obtained by conversion from a first Jones vector based on the first measurement signal and the second measurement signal and a second Jones vector based on the third measurement signal and the fourth measurement signal, respectively, and correct the von Neumann entropy based on the entropy of the noise component.

(18) In another aspect of the present invention, the first polarization state may be horizontal polarization, and the second polarization state may be vertical polarization, the first measurement signal may be a first horizontally polarized spectral interferometric signal, the second measurement signal may be a second horizontally polarized spectral interferometric signal, the third measurement signal may be a first vertically polarized spectral interferometric signal, and the fourth measurement signal may be a second vertically polarized spectral interferometric signal.

(19) In another aspect of the present invention, the evaluation unit may calculate the temporal variation characteristic value on a per observation period interval basis, the observation period interval being longer than the predetermined period.

(20) Another aspect of the present invention may include an output processing unit that determines an evaluation value indicating an active state of the sample based on the temporal variation characteristic value.

(21) Another aspect of the present invention may include an image processing unit that generates image data having, as a signal value, an output value for the temporal variation characteristic value at the observation point using a function to provide the output value monotonically changing with respect to a change in an input value.

(22) Another aspect of the present invention relates to an evaluation method for an evaluation device including acquiring an optical coherence tomography (OCT) signal indicating a state of a biological tissue provided as a sample and acquiring a signal value based on the OCT signal at an observation point in the sample, and calculating a temporal variation characteristic value indicating a temporal variation characteristic of the signal value within a predetermined period.

(23) Another aspect of the present invention relates to a program causing a computer of an evaluation device to perform a measurement procedure of acquiring an optical coherence tomography (OCT) signal indicating a state of a biological tissue provided as a sample and acquiring a signal value based on the OCT signal at an observation point in the sample, and an evaluation procedure of calculating a temporal variation characteristic value indicating a temporal variation characteristic of the signal value within a predetermined period.

### Advantageous Effects of Invention

According to the present embodiment, it is possible to detect a minute change (fluctuation) that was overlooked in the past and to realize quantitative evaluation of dynamics of biological tissues.

### BRIEF DESCRIPTION OF DRAWINGS

FIG. 1 is a diagram illustrating an evaluation method for biological tissues according to a first embodiment.

FIG. 2 is images obtained by applying the methods of Comparative Example 1 and Examples 1 and 2 to a portion of a biological tissue.

FIG. 3A is a diagram illustrating a speckle variance (time variance of a speckle signal intensity) corresponding to the image of Example 1.

FIG. 3B is a diagram illustrating a decay speed of the OCT correlation corresponding to the image of Example 2.

FIG. 3C is a diagram illustrating an attenuation coefficient of an OCT signal intensity when the method of Example 3 is applied.

FIG. 4A is a diagram illustrating a state of life or death of a biological tissue to which the method of Example 1 was applied.

FIG. 4B is a diagram illustrating a state of life or death of a biological tissue to which the method of Example 2 was applied.

FIG. 5 is images obtained by applying the methods of Comparative Example 2 and Examples 4 and 5 to a portion of a biological tissue.

FIG. 6A is a diagram illustrating a speckle variance corresponding to the image of Example 4.

FIG. 6B is a diagram illustrating a decay speed of the OCT correlation corresponding to the image of Example 5.

FIG. 6C is a diagram illustrating an attenuation coefficient of an OCT signal intensity when the method of Example 6 is applied.

FIG. 7A is a diagram illustrating a state of life or death of a biological tissue to which the method of Example 4 was applied.

FIG. 7B is a diagram illustrating a state of life or death of a biological tissue to which the method of Example 5 was applied.

FIG. 8 is a configuration diagram illustrating an example of an OCT system according to the first embodiment.

FIG. 9 is a configuration diagram illustrating an example of an OCT system according to a second embodiment.

FIG. 10 is a block diagram illustrating a configuration example of a measurement signal processing device according to the second embodiment.

FIG. 11 is a flowchart illustrating an example of OCT signal processing according to the second embodiment.

FIG. 12 is an explanatory diagram of an observation period according to the second embodiment.

FIG. 13 is a diagram illustrating an example of a spatial distribution of the variance of birefringences.

FIG. 14 is a diagram illustrating another example of a spatial distribution of the variance of birefringences.

FIG. 15 is a diagram illustrating an example of a spatial distribution of the variance of mean local birefringences.

FIG. 16 is a diagram illustrating an example of a correlation between the variance of the birefringences and the mean local birefringences.

FIG. 17 is a diagram illustrating an example of a correlation between the variance of birefringences and the variance of logarithmic intensities.

FIG. 18 is a flowchart illustrating an example of TPU calculation processing according to the second embodiment.

FIG. 19 is a diagram illustrating an example of a spatial distribution of TPUs.

FIG. 20 is a diagram illustrating an example of a temporal change in the variance of birefringences.

FIG. 21 is a diagram illustrating another example of a temporal change in TPUs.

FIG. 22 is a diagram illustrating an example of a temporal change in dynamic contrast of birefringences.

FIG. 23 is a diagram illustrating an example of a temporal change in the variance of logarithmic intensities.

### DESCRIPTION OF EMBODIMENTS

An evaluation method according to an embodiment to which the present invention is applied will be described in detail below with reference to the drawings. Further, characteristic portions in the drawings used in the following description may be expanded and illustrated for convenience in order to facilitate understanding of the characteristics, and the dimensions, ratios and the like of each constituent component are not necessarily the same as actual ones. Further, the materials, dimensions, etc. exemplified in the following description are examples, the present invention is not limited thereto, and the present invention can be appropriately modified within the range in which the gist of the invention is not changed.

### First Embodiment

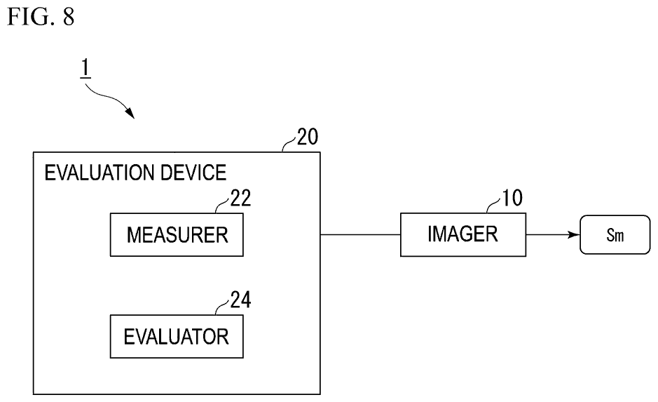

FIG. 1 is a diagram illustrating an evaluation method for biological tissues according to a first embodiment of the present invention. The purpose of the evaluation method for biological tissue according to the present embodiment is to visualize and quantitatively evaluate a fine fluctuation of a biological tissue following the next procedure in an OCT system 1 by using an evaluation device 20 for a biological tissue including an imager (imaging unit) 10 that performs optical coherence tomography (OCT) imaging for a sample Sm multiple times in a predetermined time period, a measurer (measurement unit) 22 that measures a temporal change in an OCT signal intensity obtained from an image of OCT of each operation, and an evaluator (evaluation unit) 24 that quantitatively evaluates activities of the biological tissue provided as the sample Sm based on the temporal change (see FIG. 8).

First, the imager 10 performs optical coherence tomography (OCT) imaging (measurement) for the same site at the same position on a biological tissue a plurality of times (several times to several hundreds of times that is equal to several frames to several hundreds of frames), preferably 10 frames or more, and more preferably 15 frames or more within a predetermined period (e.g., one to 15 minutes, and typically within three minutes). The predetermined period (observation period) for one OCT operation may be shorter than an observation period interval to the adjacent observation period, and may be a period in which the accuracy required for evaluation of dynamic characteristics of the sample expressed by an OCT signal obtained by the OCT operation at the time point can be ensured. More specifically, an observation period may be determined between the frame intervals and thus the frequency component of the dynamic characteristics of the sample is included in the frequency band from the lowest frequency corresponding to the observation period to the maximum frequency corresponding to the frame interval. In addition, the observation period interval may be a period in which the accuracy required for evaluation of a global change trend of the active state of the biological tissue provided as a sample can be ensured. For example, a period that is sufficiently shorter than a predetermined time of a series of processes from separating from its parent body a biological tissue serving as a sample from its parent body to cell death (apoptosis), or a series of processes required for cell death after a cause thereof occurs in a biological tissue provided as a sample in the parent body (necrosis), and the like may be determined to be an observation period interval. From the perspective of knowing characteristics of an entire sample, it is preferable to perform the same OCT operation on different sites.

Next, the measurer 22 and the evaluator 24 quantitatively evaluate activities of the biological tissue based on a temporal change in the OCT signal intensity obtained from images of OCT of each time. Specific methods of quantitative evaluation include, for example, calculation of a speckle variance (SV), an OCT correlation decay speed (OCDS), and the like, and further evaluation of calculation results.

A speckle variance indicates a variance (fluctuation) of OCT signal intensities in a short time, and can be calculated using the following formula (1).

$$\begin{matrix}[ Math . 1 ] & \\ {σ}^{2} ( x , z ) = \frac{1}{N} {\sum }_{i = 1}^{N} {[ I ( x , z , {t}_{i} ) - 〈 I 〉 ]}^{2} & ( 1 )\end{matrix}$$

In formula (1) above, x and z indicate a position on a surface of a biological tissue and a position in the depth direction from the surface, respectively, I(x, z, tᵢ) indicates an OCT signal intensity at each time at each position displayed on a linear scale or logarithmic scale, <I> indicates the mean OCT signal intensity, and N indicates the number of frames of an OCT signal within a predetermined period. x and z correspond to positions of observation points corresponding to individual pixels forming an OCT image.

More specifically, first, the measurer 22 measures each of OCT signal intensities I(x, z, t₁), I(x, z, t₂), . . . I(x, z, t_{N}) for each observation point (x, z) at the time t₁, t₂, . . . t_{N} of each frame. Next, the evaluator 24 calculates the mean <I> of the OCT signal intensity of N times. Next, the evaluator 24 squares the difference between the OCT signal intensity of each time and the mean <I>. That is, the evaluator 24 calculates [I(x, z, t₁)−<I>]², [I(x, z, t₂)−<I>]², . . . [I(x, z, t_{N})−<I>]². Finally, the evaluator 24 divides the sum of the squared differences by N and thus can obtain the speckle variance σ² (x, z).

The OCT correlation decay speed indicates a speed at which the correlation coefficient of the adjacent time intervals (t, t+τ) decreases in response to an increase in a time shift amount τ, and a correlation coefficient ρ(x, z, τ) can be calculated using the following formula (2).

[Math. 2]

ρ(x,z,τ)=σ_{cov}²(x,z,τ)/σ_{I}²(x,z) (2)

In formula (2) described above, x and z indicate a position on a surface of a biological tissue and a position in the depth direction from the surface, respectively, and σ_{cov}² (x, z, τ) and σ₁² (x, z) indicate the covariance and speckle variance (variance) of OCT signal intensities I (x, z, τ) and I (x, z, t+τ), respectively. The OCT signal intensity I (x, z, t+τ) is a signal value obtained by time-shifting the OCT signal intensity I (x, z, t) by a time shift amount τ. That is, a correlation coefficient ρ (x, z, τ) corresponds to the autocorrelation function of the signal value I (x, z, t) of the OCT signal in the observation point (x, z).

More specifically, the evaluator 24 calculates [I(x, z, t)−<I>]×[I(x, z, t+τ)−<I>] as the covariance σ_{cov}² (x, z, τ) at adjacent time intervals (t, t+τ). Next, the evaluator 24 divides the covariance σ_{cov}² (x, z, τ) by the speckle variance σ² (x, z) calculated using formula (1) described above to obtain the correlation coefficient ρ (x, z, τ). Then, the evaluator 24 performs regression analysis on the obtained correlation coefficient ρ (x, z, τ) and applies the result to a predetermined function of the time shift amount τ to obtain a parameter of the function as an OCT correlation decay speed, and thus the function value of the function more closely approximates the correlation coefficient (x, z, τ). The predetermined function may be a function in which the function value given to the time shift amount is attenuated as the time shift amount increases, for example, an exponential function. When an exponential function is used, the base being the parameter is obtained to serve as an indication of the decay speed. Although linear analysis may be used as a method of regression analysis, the method is not limited to this, and non-linear analysis may be used.

As described above, the purpose of the evaluation method for biological tissues according to the present embodiment is to quantitatively evaluate the activity of a biological tissue by obtaining images of a specific site of the biological tissue a plurality of times in a short period and calculating the speckle variance, the OCT correlation decay speed, and the like. With this operation, it is possible to detect a minute change (fluctuation) that was overlooked in the past and to realize quantitative evaluation of dynamics of the biological tissue.

### EXAMPLES

The effects of the present embodiment will be more apparent from examples below.

Further the present embodiment is not limited to the following examples, and can be appropriately changed in a range that does not change the gist of the invention.

### Comparative Example 1

A tumor aggregate sample obtained by culturing cells derived from human cancer tissues in a spherical shape was subjected to OCT imaging of the related art every 4 hours.

### Example 1

A tumor aggregate sample similar to that of Comparative Example 1 was subjected to high speed OCT imaging 100 times at intervals of 13 ms every two hours, as an example. The reason for setting the observation period interval to 2 hours is that the period is sufficient to capture the change trend of the activity state in the period from the removal of the tumor aggregate tissue provided as a sample from the living body to the death (typically, approximately 1 to 3 days). In addition, the reason for setting a single observation period to 100 times of imaging at intervals of 13 ms, that is, 1.3 seconds, is that the observation period is sufficiently shorter than the observation period interval and is sufficient for capturing the temporal change of optical characteristics in accordance with movement of the cells constituting the tissue or activities occurring within the cells (e.g., several Hz to 20 Hz). Next, the speckle variance was calculated based on the temporal change in the OCT signal intensity obtained from the images of OCT of each time.

### Example 2

A tumor aggregate sample similar to that of Comparative Example 1 was subjected to high speed OCT imaging similar to that of Example 1. Next, the OCT correlation decay speed was calculated based on the temporal change in the OCT signal intensity obtained from the images of OCT of each time.

### Example 3

A tumor aggregate sample similar to that of Comparative Example 1 was subjected to high speed OCT imaging similar to that of Example 1. Subsequently, the attenuation coefficient (AC) of the OCT signal intensity in the depth direction was calculated based on the OCT signal intensity obtained from the images of OCT of each time.

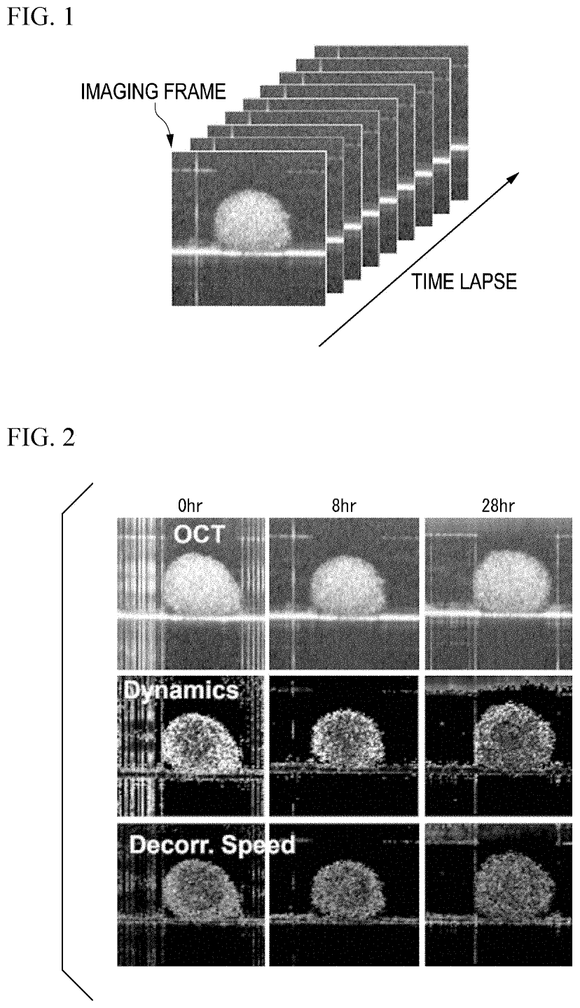

FIG. 2 is the images of the tumor aggregate samples obtained by applying the methods of Comparative Example 1 and Examples 1 and 2 thereto. The upper, middle, and lower images correspond to Comparative Example 1 and Examples 1 and 2, respectively. The numerical values of 0 hr, 8 hr, and 28 hr indicate the time that has elapsed from the time point at which the sample was cut out from the human body and started to be cultured. In the image of Comparative Example 1, no change in the state of the tumor aggregate sample according to the elapse of time is observed. On the other hand, in the images of Examples 1 and 2, there is a dark portion at the center in the initial stages and a bright portion around the center, but the bright portion seems to become gradually darker according to the elapse of time. Because it is believed that the dark portion represents a dead state and the bright portion represents a living state, it is possible to confirm the state of life or death of the tumor aggregate sample from the change in these images.

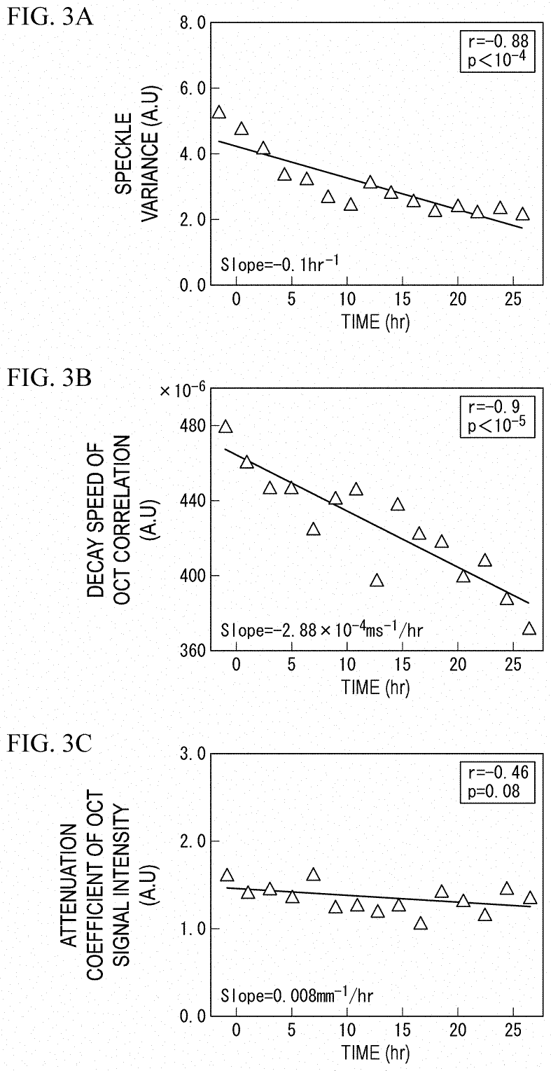

FIG. 3A and FIG. 3B are diagrams showing temporal changes in the speckle variances and OCT correlation decay speeds calculated for individual observation periods from the samples shown in the images of Examples 1 and 2 of FIG. 2, respectively. FIG. 3C is a diagram showing attenuation coefficients of the OCT signal intensity obtained when the method of Example 3 is applied. The horizontal axes of FIG. 3A, FIG. 3B, and FIG. 3C all represent the time that elapsed from the cutting out of the tumor aggregate sample, and the vertical axes represent the mean values of the speckle variance, the OCT correlation decay speed, and the attenuation coefficient of the OCT signal intensity of the entire tumor aggregate sample, respectively. In all of the drawings, the trend of decreasing mean values is seen over time. From the comparison of these three drawings, it is believed that, since the slope of the OCT correlation decay speed is the highest, it is the most suitable for quantitatively evaluating the tumor sample. In addition, the slope of the speckle variance is the next highest, which is more significantly higher than the slope of the attenuation coefficient of the OCT signal intensity proposed in the related art. Further, A.U. indicates an arbitrary unit.

Further, the image of the first time among the OCT images of a plurality of times does not correctly reflect the attenuation trend of the correlation coefficients with respect to the sample in the subsequent images, and causes a significant difference from the estimation value estimated from the attenuation trend, and thus it is preferable in quantitative evaluation to exclude the correlation coefficient for the case where the time shift amount τ is zero and calculate the OCT correlation decay speed using the correlation coefficient for a non-zero time shift amount τ that is not zero.

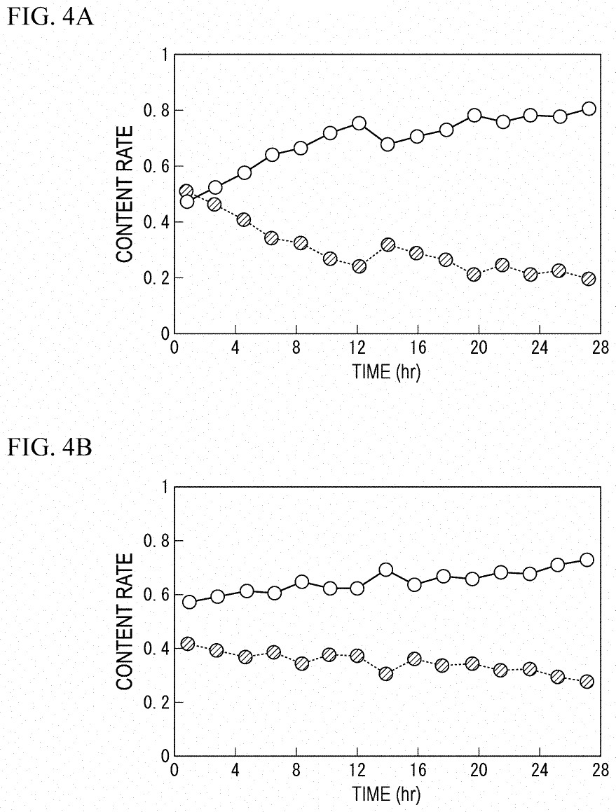

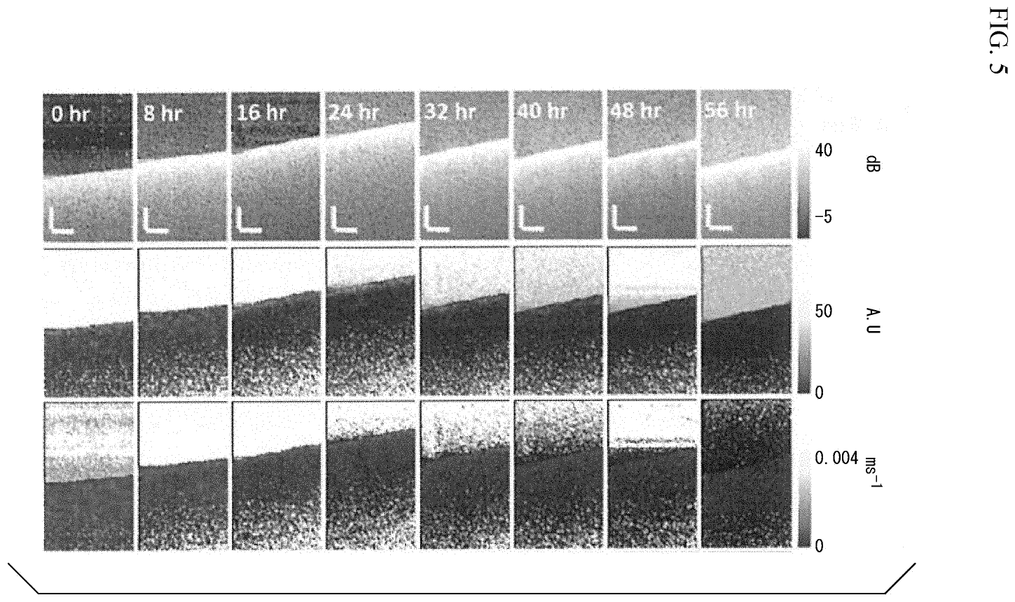

FIG. 4A and FIG. 4B are diagrams illustrating a state of life and death of a biological tissue to which the methods of Examples 1 and 2 were applied, respectively. All of the horizontal axes in FIG. 4A and FIG. 4B represent elapsed time, and the vertical axes represent content of living cells or dead cells. The boundary between life and death was set to 3.0 in Example 1 (SV) and 5×10⁴ ms⁻¹ in Example 2 (OCDS). In both graphs, there is a tendency that the number of living cells decreases and the number of dead cells increases over time.

### Comparative Example 2

A liver sample of a mouse cut out as a portion of a biological tissue was subjected to OCT imaging of the past on an hourly basis.

### Example 4

A liver sample similar to that of Comparative Example 2 was subjected to high speed OCT imaging 100 times at intervals of 10 ms on an hourly basis. Next, the speckle variance was calculated based on the temporal change in the OCT signal intensity obtained from the images of OCT of each time.

### Example 5

A liver sample similar to that of Comparative Example 2 was subjected to high speed OCT imaging similar to that of Example 4. Next, the OCT correlation decay speed was calculated based on the temporal change in the OCT signal intensity obtained from the images of OCT of each time.

### Example 6

A liver sample similar to that of Comparative Example 2 was subjected to high speed OCT imaging similar to that of Example 4. Next, the attenuation coefficient (AC) of the OCT signal intensity in the depth direction was calculated based on the OCT signal intensity obtained from the image of OCT of each time.

FIG. 5 is the images of the liver samples obtained by applying the methods of Comparative Example 2 and Examples 4 and 5 thereto. The upper, middle, and lower images correspond to Comparative Example 2 and Examples 4 and 5, respectively. The numerical values of 0 hr, 8 hr, 16 hr, and the like indicate the time that has elapsed from the time point at which the sample was cut out from the mouse serving as the mother. In the image of Comparative Example 2, no change in the state of the liver sample according to the elapse of time is observed. On the other hand, in the images of Examples 4 and 5, there is a dark portion at the lower part in the initial stages and a bright portion at the upper part, but the bright portion seems to become gradually darker over time. Because it is believed that the dark portion represents a dead state and the bright portion represents a living state, it is possible to confirm the state of life or death of the liver sample from the change in these images.

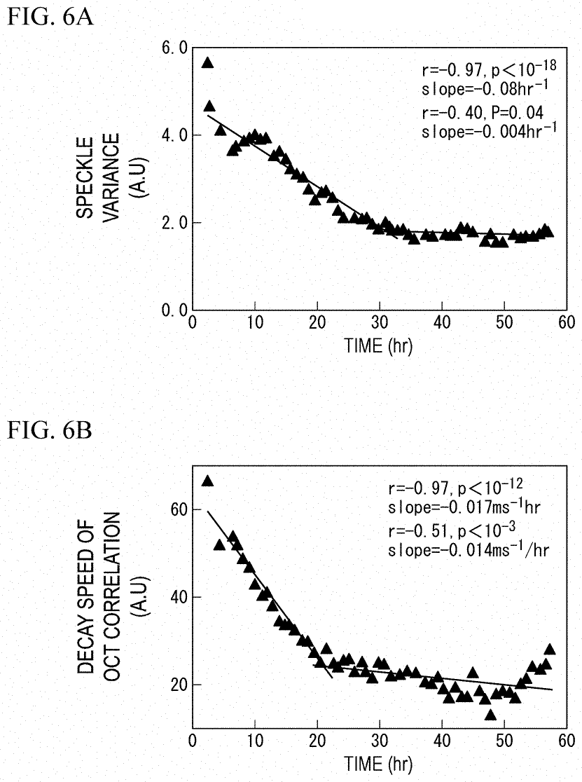

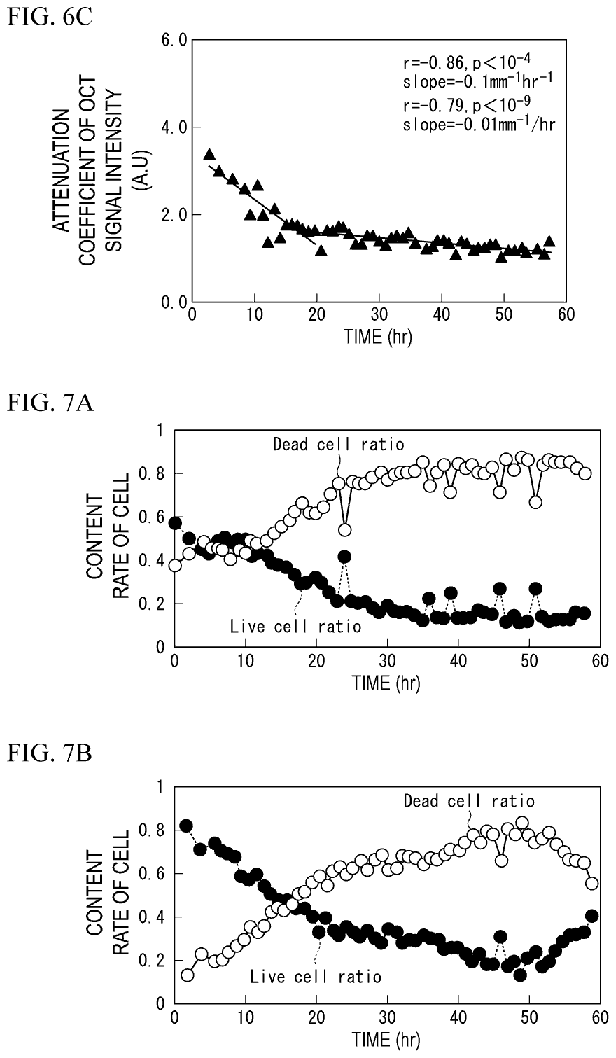

FIG. 6A and FIG. 6B are diagrams showing the speckle variances and OCT correlation decay speeds calculated for individual observation periods from the samples shown in the images of Examples 4 and 5, respectively. FIG. 6C is a diagram showing attenuation coefficients of the OCT signal intensity obtained when the method of Example 6 is applied. The horizontal axes of FIG. 6A, FIG. 6B, and FIG. 6C all represent the time that elapsed from the cutting out of the liver sample, and the vertical axes represent the mean values of the speckle variance, the OCT correlation decay speed, and the attenuation coefficient of the entire tumor liver sample, respectively. In all of the drawings, the trend of decreasing mean values with slopes in two different stages is seen over time. From the comparison of these three drawings, it is believed that, since the slope of the OCT correlation decay speed is the highest, it is the most suitable for quantitatively evaluating the tumor sample. In addition, the slope of the speckle variance is the next highest, which is more significantly higher than the slope of the attenuation coefficient of the OCT signal intensity proposed in the related art.

Further, the image of the first time among the OCT images of a plurality of times does not correctly reflect the attenuation trend of the correlation coefficients with respect to the sample in the subsequent images, and causes a significant difference from the estimation value estimated from the attenuation trend, and thus it is preferable in quantitative evaluation to calculate the OCT correlation decay speed, excluding the case where τ is zero.

FIG. 7A and FIG. 7B are diagrams illustrating a state of life or death of a biological tissue to which the methods of Examples 4 and 5 were applied, respectively. All of the horizontal axes in FIG. 7A and FIG. 7B represent elapsed time, and the vertical axes represent content of living cells or dead cells. The boundary between life and death was set to 3.0 in Example 4 (SV) and 5×10⁴ ms⁻¹ in Example 5 (OCDS). In both graphs, there is a tendency that the number of living cells decreases and the number of dead cells increases over time. According to the present embodiment, a temporal variation characteristic value such as a speckle variance (SV), an OCT correlation decay speed (OCDS), and the like is calculated for each observation period to observe samples for a longer period, and thus it is possible to observe the dynamics related to life or death of cells.

### Second Embodiment

Next, a second embodiment of the present invention will be described.

According to the analytical technique referred to as dynamic OCT described above, it is possible to express local activities of endogenous scattering factors forming biological tissues such as cells. However, in the technique described in Non-Patent Literature 1, it is difficult to evaluate activities of a living body correctly because the technique has poor quantitativeness in expressed local activities. On the other hand, in applications such as diabetes research and cardiovascular research, the application of dynamic imaging is expected to implement a visualization technique having specificity to specific tissues such as microblood vessels, lymphatic vessels, and myocardium. In order to quantitatively evaluate the activity of a biological tissue, it is also conceivable to perform OCT measurement multiple times at the same location of the biological tissue and analyze the temporal change of OCT signal intensity obtained in the measurement of each time. However, because all minute movements made in the tissue are reflected in the temporal change of the OCT signal, it is not possible to quantitatively evaluate the activity of a specific tissue simply by analyzing the temporal change of the signal intensity. The present embodiment has been proposed in view of this point.

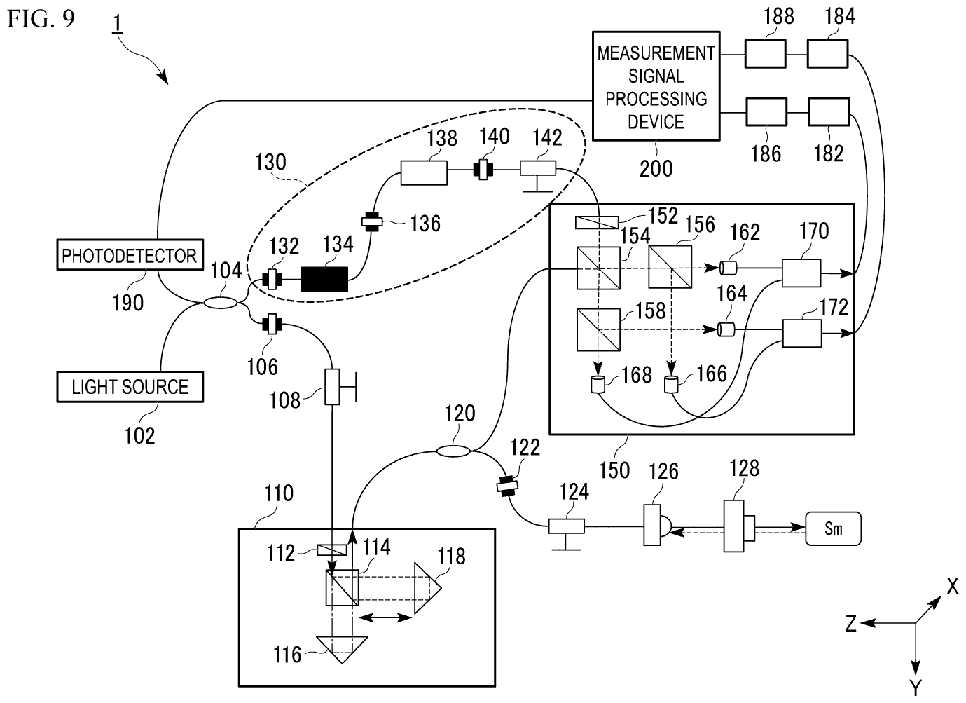

FIG. 9 is a configuration diagram illustrating an example of an OCT system 1 according to the present embodiment. The OCT system 1 constitutes PS-OCT. PS-OCT includes an optical system that radiates incident light in a known polarization state to a sample Sm for acquiring interferometric light obtained by causing reflected light reflected from the sample Sm to interfere with reference light. In addition, the OCT system 1 includes a measurement signal processing device 200 that analyzes characteristics of a change from the polarization state of the interferometric light acquired by the optical system to the polarization state of the sample Sm. The measurement signal processing device 200 functions as an evaluation device that analyzes a state of a biological tissue provided as the sample Sm using an OCT signal. The measurement signal processing device 200 generates an image in which the analyzed characteristics of a change are visualized.

An object to be observed that is provided as a sample Sm is primarily a part of a living body of a human or an animal. More specifically, it may be any of an ocular fundus, a blood vessel, a tooth, a subcutaneous tissue, and the like. This makes it possible to measure or observe the state inside the sample Sm in a non-invasive manner. Thus, the device is expected to be applied to diagnosis of in vivo tissues, for example, microvessels of an ocular fundus, lymphatic vessels, myocardium, and the like.

The OCT system 1 exemplified in FIG. 9 is an observation system to which a wavelength-swept type OCT (swept source-OCT or SS-OCT) for sweeping the wavelength of light generated by a light source 102 to obtain a spectral interferometric signal is applied. The OCT system 1 splits light emitted from the light source 102 to be incident on a probe arm (described below) and a reference arm 130. The OCT system 1 separates the light split into the probe arm into a horizontal polarization component and a vertical polarization component, radiates light including polarization components having different optical path lengths therebetween to a sample Sm to be measured while scanning the sample (B-scan), and acquires reflected light reflected from the sample Sm (object light). The OCT system 1 acquires interferometric light by interfering with the reference light split to the reference arm 130 and reflected light that is a component obtained from reflection or scattering of light from the sample Sm or from both of the phenomena. Further, in the present application, the direction in which light is radiated to the sample Sm is referred to as a depth direction. The acquisition of a measurement signal by scanning the observation point in the depth direction of the sample Sm is referred to as A-scan. In SS-OCT, A-scan is achieved by using a wavelength sweeping light source. B-scan refers to scanning of a sample Sm in a direction perpendicular to the depth direction.

The OCT system 1 includes the light source 102, a coupler 104, a polarization delay unit 110, a circulator 120, a probe 128, the reference arm 130, a polarization diversity detection unit 150, a photodetector 190, and the measurement signal processing device 200. The light source 102, the coupler 104, the polarization delay unit 110, the circulator 120, the probe 128, the polarization diversity detection unit 150, and the photodetector 190 are components constituting an optical system. The components of the optical system are coupled using optical fibers as optical paths.

The light source 102 is a wavelength swept source that generates light having a wavelength to be periodically swept within a predetermined wavelength width (e.g., 40 to 120 nm). The light source 102 has a wavelength of near-infrared (e.g., 1000 to 1400 nm), for example, superluminescent diode (SLD), or the like. Light generated by the light source 102 is incident on the coupler 104.

The coupler 104 separates the light incident from the light source 102 into two systems of the probe arm and the reference arm 130 at a predetermined intensity ratio. The percentages of the light intensity for the probe arm and the light intensity for the reference arm 130 are, for example, 90% and 10%. The light supplied to the probe arm is supplied to the polarization diversity detection unit (PDDU) 150. The probe arm is a path formed by a fiber collimator 106, a polarization controller 108, the polarization delay unit (PDU) 110, the circulator 120, a fiber collimator 122, a polarization controller 124, an objective lens 126, and the probe 128 connected in this order. The probe arm is also called a sample arm or a measuring arm. Light supplied to the probe arm is incident on the polarization delay unit 110 via the fiber collimator 106 and the polarization controller 108. Light of another system is incident on the PPDU 150 via the reference arm 130. Further, the polarization controller 108 amplifies the intensity of the incident light to a predetermined sufficient intensity, and then emits the amplified light.

The PDU 110 includes a linear polarizer 112, a polarization beam splitter (PBS) 114, and two right-angle prisms (RAPs) 116 and 118. The PDU 110 separates the incident light into a horizontal polarization component and a vertical polarization component as components having two polarization states orthogonal to each other, and supplies the light obtained by combining the separated components to the circulator 120.

The linear polarizer 112 converts the light incident from the coupler 104 in the polarization state into linearly polarized light and emits the converted light to the PBS 114. To equalize the horizontal polarization component and the vertical polarization component of the emitted light, the polarization angle of the linear polarizer 112 is set to 45°. The PBS 114 has a reflective layer whose surface is arranged in the direction at an incident angle of 45°, the vertical polarization component of the incident light incident on the reflective layer from the linear polarizer 112 is transmitted as transmitted light, and the horizontal polarization component thereof reflects on the surface of the reflective layer as reflected light. The reflected light including the horizontal polarization component and the transmitted light including the vertical polarization component from the PBS 114 are incident on the RAPs 116 and 118, respectively. On the other hand, the reflective layer of the PBS 114 combines the transmitted light obtained by transmitting incident light including the horizontal polarization component that is incident on the reflective layer from the RAP 116 with the reflected light with respect to the incident light including the vertical polarization component incident from the PBS 118, and emits the combined light to the circulator 120.

The RAPs 116 and 118 each have a shape in which a cross section parallel to the optical path is an isosceles right triangle, and a bottom side of the isosceles right triangle is disposed to be orthogonal to the optical path from the PBS 114. The light incident from the PBS 114 is transmitted through the side face parallel to the base of the isosceles right triangle and is reflected on one side face parallel to one of the two sides facing the base, and the reflected light is reflected on the side face parallel to the other side to return to the side face parallel to the bottom to be transmitted through the side face and incident on the PBS 114. Further, the position of the RAP 118 is adjusted in advance and thus the optical path length between the PBS 114 and the RAP 116 and the optical path length between the PBS 114 and the RAP 118 are significantly different. In this way, the horizontal polarization component and the vertical polarization component that are incident on the sample Sm from the PBS 114 are emitted in a superimposed manner with a predetermined phase difference between each other.

The circulator 120 emits light incident from the PDU 110 through the fiber collimator 122 and the polarization controller 124 to the objective lens 126. The objective lens 126 condenses light incident on the lens to radiate the light to the sample Sm via the probe 128. The light obtained from reflection or scattering from the sample Sm or both of the phenomena is converted to parallel light by the objective lens 126 via the probe 128, passes through the polarization controller 124 and the fiber collimator 122 to return to the circulator 120, and is incident on the PPDU 150 as a measurement beam.

The reference arm 130 is a path formed by a fiber collimator 132, a fiber Bragg grating (FBG) 134, a fiber collimator 136, a delay line 138, a fiber collimator 140, and a polarization controller 142 connected in this order.

The FBG 134 reflects components of incident light of a specific wavelength as reflected light and transmits the rest components and causes the components to be incident on the delay line 138 via the fiber collimator 136 as transmitted light. The reflected light from the FBG 134 returns to the coupler 104 via the fiber collimator 132 and is incident from the coupler 104 to the photodetector 190. The band of components reflected from the FBG 134 is sufficiently narrower than the band of light generated by the light source 102. The photodetector 190 detects the intensity of the reflected light from the FBG 136 and outputs the intensity signal indicating the detected intensity to the measurement signal processing device 200 as a trigger signal. The trigger signal is used as a trigger for A-scan. Although the wavelength of the light generated by the light source 102 changes periodically within the range of a predetermined wavelength width, the timing at which the wavelength becomes a predetermined wavelength is detected by the photodetector 190, and an optical system control unit 212 resets A-scan at that timing. For example, a lower limit of the wavelength width for the photodetector 190 is preset as the wavelength to be detected. The reason for this is that the depth of an observation point to be observed is determined by the wavelength of probe light in SS-OCT.

The delay line 138 delays incident light incident from the FBG 134 and emits the delayed light to the PPDU 150 via the fiber collimator 140 and the polarization controller 142. The delay line 138 changes a delay amount of incident light and adjusts a delay amount, and thus the optical path length of the probe arm and the optical path length of the reference arm 130 are equal to each other. Further, the polarization controller 142 adjusts the intensity of incident light to a predetermined intensity, and emits light with the adjusted intensity.

The PPDU 150 includes a linear polarizer 152, a non-polarization beam splitter (NPBS) 154, two PBSs 156 and 158, four optical receivers 162, 164, 166 and 168, and two balanced polarization detectors (BPD) 170 and 172.

The linear polarizer 152 converts light incident from the reference arm 130 in a polarization state into linearly polarized light and emits the converted light to the NPBS 154. A polarization angle of the linear polarizer 152 is set to 45°. The NPBS 154 combines incident light incident from the reference arm 130 via the linear polarizer 152 and incident light incident from the probe arm. The NPBS 154 has a reflective layer whose surface is disposed in the direction in which the incident angle is 45° with respect to each of incident light from the reference arm 130 and incident light from the probe arm. The reflective layer combines transmitted light obtained by transmitting the incident light from the reference arm 130 and reflected light obtained by reflecting incident light from the probe arm, and emits interferometric light obtained from the combination to the PBS 158. The reflective layer combines reflected light obtained by transmitting the incident light from the reference arm 130 and transmitted light obtained by transmitting the incident light from the probe arm, and emits interferometric light obtained from the combination to the PBS 156.

The PBS 156 separates the interferometric light incident from the NPBS 154 into a horizontally polarized component and a vertically polarized component, and emits the separated horizontally polarized component and vertically polarized component to the optical receivers 162 and 166, respectively. The optical receivers 162 and 166 receive the horizontally polarized component and the vertically polarized component incident from the PBS 156, respectively, and guide the components to the BPDs 170 and 172 as a first horizontally polarized component and a first vertically polarized component. The first horizontally polarized component and the first vertically polarized component correspond to the horizontally polarized component and the vertically polarized component of the interferometric light based on the horizontally polarized component incident on the sample Sm.

The PBS 158 separates the light incident from the NPBS 154 into a horizontally polarized component and a vertically polarized component, and emits the separated horizontally polarized component and vertically polarized component to the optical receivers 164 and 168, respectively. The optical receivers 164 and 168 receive the horizontally polarized component and the vertically polarized component incident from the PBS 158, respectively, and guide the components to the BPDs 170 and 172 as a second horizontally polarized component and a second vertically polarized component. The second horizontally polarized component and the second vertically polarized component correspond to a horizontally polarized component and a vertically polarized component of the interferometric light based on the vertically polarized component incident on the sample Sm.

The BPD 170 detects the first horizontally polarized component and the second horizontally polarized component guided from the optical receivers 162 and 166, and converts the components into a first horizontally polarized spectral interferometric signal and a second horizontally polarized spectral interferometric signal that are analog electrical signals indicating the intensities of the detected first horizontally polarized component and second horizontally polarized component, respectively. The BPD 170 has the generated first horizontally polarized spectral interferometric signal and second horizontally polarized spectral interferometric signal pass through a low pass filter (LPF) 182 and a high pass filter (HPF) 186 to be output to the measurement signal processing device 200.

The BPD 172 detects the first vertically polarized component and the second vertically polarized component guided from the optical receivers 164 and 168, and converts the components into a first vertically polarized spectral interferometric signal and a second vertically polarized spectral interferometric signal that are analog electrical signals indicating the intensities of the detected first vertically polarized component and second vertically polarized component, respectively. The BPD 172 has the generated first vertically polarized spectral interferometric signal and second vertically polarized spectral interferometric signal pass through a low pass filter 184 and a high pass filter 188 to be output to the measurement signal processing device 200. Each of the first horizontally polarized spectral interferometric signal, the second horizontally polarized spectral interferometric signal, the first vertically polarized spectral interferometric signal, and the second vertically polarized spectral interferometric signal are used to generate an OCT image of one frame to realize Jones matrix OCT (JM-OCT).

Further, the BPDs 170 and 172 samples the width of the wavelength of the light emitted by the light source 102 to a signal value of a predetermined number of samples (e.g., 400 to 2000 samples) at a predetermined sampling frequency for each time of A-scan. Each of the LPFs 182 and 184 and the HPFs 186 and 188 is, for example, a Chebyshev filter. The cutoff frequency of the LPFs 182 and 184 is, for example, 62 MHz. The cutoff frequency of the HPFs 186 and 188 is, for example, 1 MHz.

Measurement Signal Processing Device

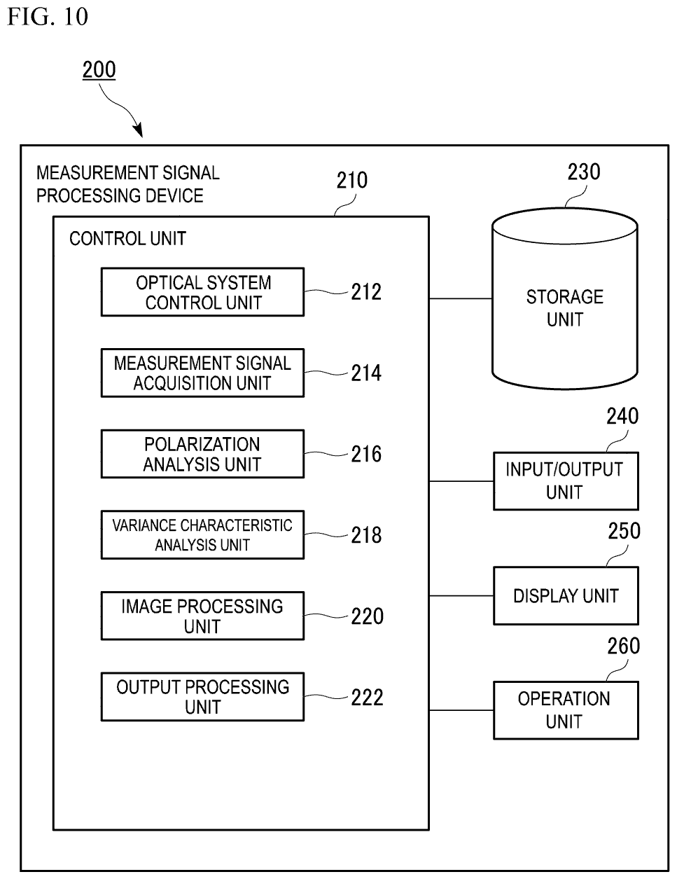

Next, a configuration example of the measurement signal processing device 200 according to the present embodiment will be described. FIG. 10 is a block diagram illustrating a configuration example of the measurement signal processing device 200 according to the present embodiment. The measurement signal processing device 200 analyzes polarization characteristics at an observation point in the sample from each of the first horizontally polarized spectral interferometric signal, the second horizontally polarized spectral interferometric signal, the first vertically polarized spectral interferometric signal, and the second vertically polarized spectral interferometric signal input from the PPDU 150, analyzes temporal variation characteristics of the polarization characteristic values indicating the analyzed polarization characteristics, and determines a temporal variation characteristic value indicating the analyzed temporal variation characteristic. The measurement signal processing device 200 may convert the color or grayscale corresponding to the temporal variation characteristic value determined for each of the observation points into signal values, generate image data for each pixel corresponding to the observation points using the converted signal values, and output the generated image data.

The measurement signal processing device 200 includes a control unit 210, a storage unit 230, an input/output unit 240, a display unit 250, and an operation unit 260. Some or all functions of the control unit 210 are realized as a computer including a processor such as a central processing unit (CPU), for example. The processor reads a program stored in the storage unit 230 in advance, and performs processing indicated by a command described in the read program to provide the function. In the present application, the processing indicated by the command described in the program may be referred to as executing the program, execution of the program, or the like. Some or all of the control unit 210 is not limited to a general-purpose hardware such as a processor, and may include dedicated hardware such as a large scale integration (LSI), an application specific integrated circuit (ASIC), or the like.

The control unit 210 includes an optical system control unit 212, a measurement signal acquisition unit 214, a polarization analysis unit 216, a variance characteristic analysis unit 218, an image processing unit 220, and an output processing unit 222. The measurement signal acquisition unit 214 and the polarization analysis unit 216 according to the present embodiment acquire an OCT signal, and function as a measurement unit that acquires a signal value based on the acquired OCT signal for each observation point of the sample. The variance characteristic analysis unit 218 functions as an evaluation unit for calculating a temporal variation characteristic value indicating temporal variation characteristic of the acquired signal value for each predetermined period.

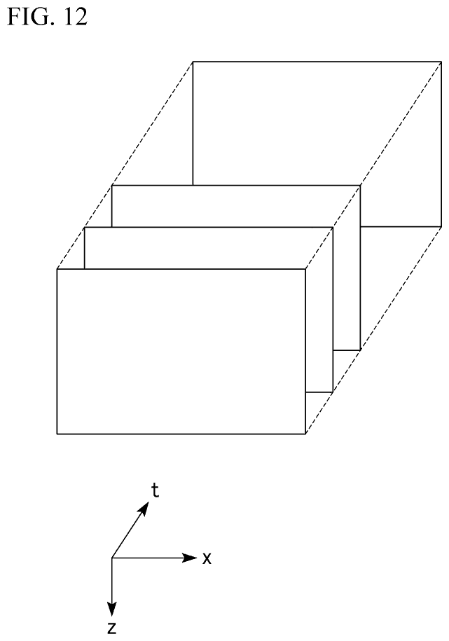

The optical system control unit 212 drives a drive mechanism that makes a position of the probe variable, and scans an observation point of the sample Sm (B-scan). The optical system control unit 212 scans the observation point of the sample Sm in a predetermined direction that intersects the depth direction (hereinafter, the z direction) of the sample Sm (e.g., the x direction on the front of the sample Sm orthogonal to the depth direction). The optical system control unit 212 outputs a control signal indicating movement of the position of the probe 128 in that direction to the drive mechanism when a trigger signal input from the photodetector 190 is equal to or greater than a predetermined intensity, for example. A distance of the movement corresponds to a preset interval between observation points in the x direction. In addition, at this time, the optical system control unit 212 returns the position of the probe 128 to the reference point each time the number of observation points in the x direction from the reference point reaches a predetermined number (the number of lines). Thereafter, the measurement signal acquisition unit 214 acquires the measurement signal of the next frame. The repetition of the measurement signal causes the measurement signal to be accumulated per frame over time. In the example illustrated in FIG. 12, the x direction, the z direction, and a time t are shown to face to the right, downward, and to the upper right, respectively, and each frame is illustrated in an individual rectangle.

The measurement signal acquisition unit 214 receives the input of the first horizontally polarized spectral interferometric signal, the second horizontally polarized spectral interferometric signal, the first horizontally polarized spectral interferometric signal, and the second vertically polarized spectral interferometric signal as measurement signals from the PPDU 150 via the input/output unit 240. The measurement signal acquisition unit 214 performs a Fourier transform on the first horizontally polarized spectral interferometric signal, the second horizontally polarized spectral interferometric signal, the first vertically polarized spectral interferometric signal, and the second vertically polarized spectral interferometric signal, and calculates a first horizontally polarized OCT signal, a second horizontally polarized OCT signal, a first vertically polarized OCT signal, and a second vertically polarized OCT signal each indicating a complex amplitude for each observation point. The measurement signal acquisition unit 214 outputs the calculated first horizontally polarized OCT signal, second horizontally polarized OCT signal, first vertically polarized OCT signal, and second vertically polarized OCT signal to the polarization analysis unit 216.

The polarization analysis unit 216 calculates, for each observation point within the sample Sm, a polarization characteristic value indicating the polarization characteristics at the observation points based on the first horizontally polarized OCT signal, the second horizontally polarized OCT signal, the first vertically polarized OCT signal, and the second vertically polarized OCT signal input from the measurement signal acquisition unit 214. The polarization analysis unit 216 configures a Jones matrix indicating the polarization characteristics of the light waves to be measured, and calculates a predetermined polarization characteristic value as an index value representing the polarization characteristics from the configured Jones matrix.

The Jones matrix is a matrix with two rows and two columns indicating a change in a polarization state. A first Jones vector and a second Jones vector are arranged in the first and second columns of the Jones matrix, respectively. The first Jones vector and the second Jones vector are acquired by using incident light having polarization components orthogonal to each other. That is, the first Jones vector is a two-dimensional vector including complex amplitudes each indicated by the first horizontally polarized OCT signal and first vertically polarized OCT signal as elements. The second Jones vector is a two-dimensional vector including complex amplitudes each indicated by the second horizontally polarized OCT signal and second vertically polarized OCT signal as elements. Each column of the Jones matrix corresponds to a polarization state related to incidence, each row corresponds to a detected polarization state. The polarization analysis unit 216 outputs the calculated polarization characteristic value for each observation point to the variance characteristic analysis unit 218. Further, in the present application, the Jones matrix directly composed of the OCT signals or the conversion coefficients in the frequency domain thereof at this stage may be referred to as a measured Jones matrix.

The variance characteristic analysis unit 218 calculates a predetermined temporal variation characteristic value as an index value indicating a temporal variation characteristic in an observation period that is a predetermined period set in advance for the polarization characteristic value input from the polarization analysis unit 216. The observation period is, for example, typically about 150 to 600 frames when the frame rate is 60 frames/second. The variance characteristic analysis unit 218 outputs the calculated temporal variation characteristic value to the image processing unit 220. Further, examples of the polarization characteristic value and the temporal variation characteristic value will be described below.

The image processing unit 220 converts the temporal variation characteristic value input from the variance characteristic analysis unit 218 into a pixel value within a predetermined value range that can be expressed by a bit depth for each pixel using a predetermined function. The image processing unit 220 calculates a pixel value by adding a predetermined offset value to the multiplication value obtained by multiplying a value of a sigmoid function for a temporal variation characteristic value by a predetermined multiple. The function for converting the temporal variation characteristic value into a pixel value is not limited to a Sigmoid function, and a function is available as long as a function value obtained from an input value monotonically increases or decreases with respect to an increase in the input value, such as a linear function or a logarithmic function. The image processing unit 220 generates output image data indicating the pixel value converted for each of the observation points, and outputs the generated output image data to the display unit 250. The image processing unit 220 may store the output image data in the storage unit 230 in accordance with a control signal input from the output processing unit 222.

The output processing unit 222 controls the generation or output of the output image data indicating the display image based on an operation signal input from the operation unit 260. The operation signal indicates, for example, necessity of display or storage of a display image, an observation target region, or the like as a parameter. In a case where the control unit 210 has the capability of calculating a plurality of types of temporal variation characteristic values, calculation or the type of a display target among a plurality of types of temporal variation characteristic values may be indicated using operation signals. The output processing unit 222 may cause the display unit to display a setting screen for guiding a parameter that can be set in an operation, a parameter setting, and a parameter to be set, and may configure a user interface related to image display.

For example, when an operation signal indicating necessity of display of a display image is input, the output processing unit 222 outputs a control signal indicating whether display is needed to the image processing unit 220. The image processing unit 220 outputs the output image data to the display unit when the control signal indicating necessary of the display is input from the output processing unit 222, and does not output the output image data to the display unit when the control signal indicating unnecessity of the display is input from the output processing unit 222.

When an operation signal indicating an observation target region is input, the output processing unit 222 outputs a control signal indicating the range of an x coordinate or a y coordinate in the observation target region to the measurement signal acquisition unit 214. The measurement signal acquisition unit 214 performs B-scan in the range indicated by the control signal input from the output processing unit 222. For the observation target region, for example, the range of a surface of the sample Sm is defined as a parameter.

In addition to the above-described program, the storage unit 230 stores various types of data to be used in processing performed by the control unit 210 and various types of data acquired by the control unit 210. The storage unit 230 includes a non-volatile (non-transitory) storage medium, for example, a read only memory (ROM), a flash memory, a hard disk drive (HDD), or the like. The storage unit 230 includes a volatile storage medium, for example, a random access memory (RAM), a register, or the like.

The input/output unit 240 is connected to other devices wirelessly or by wire to input and output various types of data. The input/output unit 240 includes, for example, an input/output interface. The input/output unit 240 is connected to the polarization diversity detection unit 150 and the photodetector 190, for example. The display unit 250 displays an image based on the output image data input from the control unit 210. The display unit 250 may include, for example, any of a liquid crystal display, an organic electroluminescent display, or the like.

The operation unit 260 may include a member that receives a user operation, for example, a button, a lever, a dial, a mouse, a joystick, or the like, to generate an operation signal in accordance with the received operation. The operation unit 260 outputs the acquired operation signal to the control unit 210. The operation unit 260 may be an input interface that receives an operation signal wirelessly or by wire from other devices (e.g., a mobile device such as a remote controller).

OCT Signal Processing

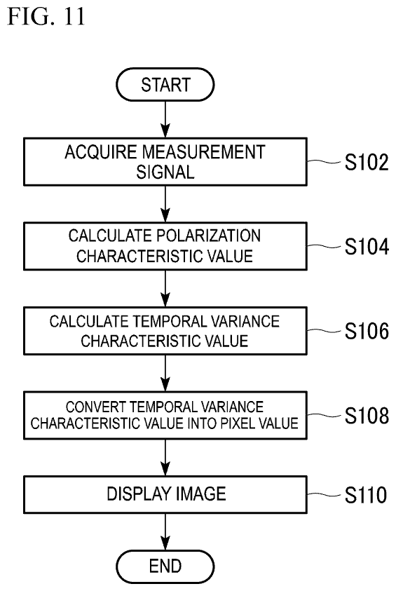

Next, an example of OCT signal processing according to the present embodiment will be described. FIG. 11 is a flowchart showing an example of OCT signal processing according to the present embodiment. (Step S102) The measurement signal acquisition unit 214 acquires a first horizontally polarized spectral interferometric signal, a second horizontally polarized spectral interferometric signal, a first vertically polarized spectral interferometric signal, and a second vertically polarized spectral interferometric signal from the optical system to calculate a first horizontally polarized OCT signal, a second horizontally polarized OCT signal, a first vertically polarized OCT signal, and a second vertically polarized OCT signal from the acquired signals. (Step S104) The polarization analysis unit 216 configures a Jones matrix for each observation point based on the first horizontally polarized OCT signal, the second horizontally polarized OCT signal, the first vertically polarized OCT signal, and the second vertically polarized OCT signal acquired from the measurement signal acquisition unit 214. The polarization analysis unit 216 calculates a predetermined polarization characteristic value from the configured Jones matrix.

(Step S106) The variance characteristic analysis unit 218 calculates a predetermined temporal variation characteristic value from the change characteristic value calculated by the polarization analysis unit 216 in the predetermined observation period. (Step S108) The image processing unit 220 converts a temporal variation characteristic value for each observation point calculated by the variance characteristic analysis unit 218 into a pixel value of a pixel corresponding to an individual observation point. (Step S110) The image processing unit 220 outputs the output image data indicating the transformed pixel value to the display unit 250 (image display). Thus, the display unit 250 visualizes a distribution of the temporal variation characteristic values in the observation target region, the values indicating the polarization characteristics in the observation target region.

Example of Polarization Characteristic Value and Temporal Variation Characteristic Value

Next, an example of a polarization characteristic value and a temporal variation characteristic value will be described. The polarization analysis unit 216 calculates a phase retardation, for example, as a polarization characteristic value. The phase retardation is the phase difference between an ordinary ray and an extraordinary ray caused by birefringence. That is, ordinary rays and extraordinary rays have polarization directions orthogonal to each other with respect to an optical axis of a sample, and are transmitted through the sample at different speeds. A phase retardation cumulated from a surface to an observation point of interest in the sample in the phase retardation is called a cumulative phase retardation (CPR). The polarization analysis unit 216 calculates a cumulative Jones matrix J_{cz} for the observation point by multiplying an inverse matrix Jₘ₀⁻¹ of a Jones matrix at a position on a sample surface corresponding to the observation point in a depth z by a measurement Jones matrix J_{mz} at the observation point in the depth z as shown in formula (3). The cumulative Jones matrix J_{cz} indicates a change in the polarization state from the sample surface to the observation point in the depth z.

[Math. 3]

J_{cz}=J_{mz}Jₘ₀⁻¹ (3)

The polarization analysis unit 216 can calculates a phase difference arg(λ_{c1} λ_{c2}*) of two eigenvalues λ_{c1} and λ_{c2} obtained by performing eigendecomposition on the cumulative Jones matrix J_{cz} as CPRs. Further, arg ( . . . ) indicates a deflection angle of a complex . . . , and ˜* indicates a complex conjugation to a complex ˜.

Further, the polarization analysis unit 216 may calculate a local phase retardation (LPR) that is a polarization phase retardation in a local depth region as another example of a polarization characteristic value. More specifically, the polarization analysis unit 216 calculates a local Jones matrix J₁₁₂ for the observation points in depths z₁ and z₂ obtained by multiplying an inverse matrix J_{cz1}⁻¹ of a cumulative Jones matrix for the observation point in the depth z₁ by the cumulative Jones matrix for the observation point in the depth z₂ by J_{cz2} as shown in formula (4). The local Jones matrix J₁₁₂ indicates a change in the polarization state from the observation point in the depth z₁ to the observation point in the depth z₂.

[Math. 4]

Jₗ₁₂=J_{cz2}J_{cz1}⁻¹ (4)

A thickness Δz₁₂ from the observation point in the depth z₁ to the observation point in the depth z₂ may be, for example, a thickness of a tissue to be observed indicated by an operation signal from the operation unit 260, or a distance between the observation points in the z direction. The polarization analysis unit 216 can calculates a phase difference arg (λ₁₁ λ₁₂*) of two eigenvalues l₁₁ and λ₁₂ of the local Jones matrix J₁₁₂ as an LPR. Further, the polarization analysis unit 216 may calculate the local Jones matrix J₁₁₂ for the observation points in the depths z₁ and z₂ by multiplying an inverse matrix J_{mz1}⁻¹ of a measurement Jones matrix for the observation point in the depth z₁ by a measurement Jones matrix J_{mz2} for the observation point in the depth z₂.

In addition, the polarization analysis unit 216 may calculate a birefringence b₁₂ at the observation points in depths z₁ and z₂ by dividing the LPR by 2k₀Δz₁₂ as shown in formula (5) as another example of a polarization characteristic value. Where k₀ indicates the center wavelength of incident light.

[Math. 5]

b₁₂=LPR/2k₀Δz₁₂ (5)

The variance characteristic analysis unit 218 calculates the variance of the polarization characteristic value as an example of a temporal variation characteristic value within a predetermined observation period. The calculated temporal variation characteristic value indicates the magnitude of temporal variance of the polarization state indicated by the polarization characteristic value. In formula (6), σ₁² (x, z) indicates the variance of an LPR. N, φ(x, z, tᵢ) and <φ(x, z)> indicate the number of frames, an LPR, and a time average of LPRs in a predetermined observation period, respectively. (x, z) indicates coordinates of an observation point in the x-z plane. tᵢ indicates an i-th sample time.

$$\begin{matrix}[ Math . 6 ] & \\ {σ}^{2} ( x , z ) = \frac{1}{N} {\sum }_{i = 1}^{N} {[ ϕ ( x , z , {t}_{i} ) - < ϕ ( x , z ) > ]}^{2} & ( 6 )\end{matrix}$$

Although formula (6) illustrates the variance of an LPR, the variance characteristic analysis unit 218 may calculate the variance of the CPR or the variance of the birefringence, instead of LPR. The variance characteristic analysis unit 218 may calculate the square roots of these variances as standard deviations.

The variance characteristic analysis unit 218 may calculate the variance or standard deviation of the logarithmic value of the polarization characteristic value as another example of the temporal variation characteristic value. In the course of calculating the variance or standard deviation, the logarithmic value of the time averaged value is subtracted from the logarithmic value of the polarization characteristic value at each time, so the constant that is potentially multiplied by the polarization characteristic value is eliminated. Thus, it may be applied to evaluation of a substantial temporal variation characteristic. In addition, by taking the logarithmic value, it may be applied to a comparison between different phenomena having a large scale. In formula (7), log σ₁² (x, z) indicates the variance of the logarithmic value of the LPR.

$$\begin{matrix}[ Math . 7 ] & \\ log {σ}_{l}^{2} ( x , z ) = \frac{1}{N} {\sum }_{i = 1}^{N} {[ log ( ϕ ( x , z , {t}_{i} ) ) - < log ( ( β ( x , z ) ) > ]}^{2} & ( 7 )\end{matrix}$$

Although formula (7) takes the variance of the logarithmic value of the LPR as an example, the variance characteristic analysis unit 218 may calculate the variance of the logarithmic value of the CPR or the variance of the logarithmic value of the birefringence, instead of the variance of the logarithmic value of the LPR. The variance characteristic analysis unit 218 may calculate the square roots of these variances as standard deviations.

The variance characteristic analysis unit 218 may calculate, for example, a dynamic contrast of the polarization characteristic value as a temporal variation characteristic value within a predetermined observation period. The dynamic contrast of the polarization characteristic value corresponds to a value normalized by dividing the standard deviation of the polarization characteristic value by the time average. In formula (8), φ_{d} indicates a dynamic contrast of an LPR.

$$\begin{matrix}[ Math . 8 ] & \\ {ϕ}_{d} ( x , z ) = \frac{\sqrt{\frac{1}{N} {\sum }_{i = 1}^{N} {( ϕ ( x , z , {t}_{i} ) - < ϕ ( x , z ) > )}^{2}}}{< ϕ ( x , z ) >} & ( 8 )\end{matrix}$$

Although formula (8) takes a dynamic contrast of an LPR as an example, the variance characteristic analysis unit 218 may calculate a CPR or a dynamic contrast of a birefringence, instead of an LPR. In the course of calculating a dynamic contrast, the standard deviation is normalized with the time average, and thus a dynamic contrast can be applied to the evaluation of the substantial temporal variation characteristic rather than the standard deviation.

Further, the variance characteristic analysis unit 218 may calculate a temporal polarization uniformity (TPU) as another example of the temporal variation characteristic value, A method for calculating a TPU will be described with reference to FIG. 18. First, the polarization analysis unit 216 converts a first Jones vector J₁ and a second Jones vector J₂ forming a partial space of the measurement Jones matrix J_{mz} for an observation point into a first Stokes vector S₁ and a second Stokes vector S₂, respectively, as another example of the polarization characteristic value (step S122 in FIG. 18). As shown in formula (9), the first Jones vector J₁ and the second Jones vector J₂ include an element of a first column and an element of a second column of the measurement Jones matrix J_{mz}, respectively.

$$\begin{matrix}[ Math . 9 ] & \\ {J}_{mz} = ⌊ \begin{matrix}{g}_{1 H} & {g}_{2 H} \\ {g}_{1 V} & {g}_{2 V}\end{matrix} ⌋ = [ \begin{matrix}{J}_{1} & {J}_{2}\end{matrix} ] , {J}_{1} = ⌊ \begin{matrix}{g}_{1 H} \\ {g}_{1 V}\end{matrix} ⌋ , {J}_{2} = ⌊ \begin{matrix}{g}_{2 H} \\ {g}_{2 V}\end{matrix} ⌋ & ( 9 )\end{matrix}$$

The first Stokes vector S₁ and the second Stokes vector S₂ are 4-dimensional vectors representing the polarization states indicated by the first Jones vector J₁ and the second Jones vector J₂, respectively, as shown in formula (10), and the vectors include element values of the first Jones vector J₁ and the second Jones vector J₂, respectively. In the following description, four element values s₁₀ to s₁₃ constituting the first Stokes vector J₁ and four element values s₂₀ to s₂₃ constituting the second Stokes vector J₂ will be called a zero-th to a third Stokes parameters s₁₀ to s₁₃ and a zero-th to a third Stokes parameters s₂₀ to s₂₃.

$$\begin{matrix}[ Math . 10 ] & \\ {S}_{1} = ⌊ \begin{matrix}{s}_{10} \\ {s}_{11} \\ {s}_{12} \\ {s}_{13}\end{matrix} ⌋ = ⌊ \begin{matrix}{\vert {g}_{1 H} \vert}^{2} + {\vert {g}_{1 V} \vert}^{2} \\ {\vert {g}_{1 H} \vert}^{2} - {\vert {g}_{1 V} \vert}^{2} \\ 2 Re [ {g}_{1 H} {g}_{1 V}^{*} ] \\ 2 Im [ {g}_{1 H} {g}_{1 V}^{*} ]\end{matrix} ⌋ = ⌊ \begin{matrix}{\vert {g}_{1 H} \vert}^{2} + {\vert {g}_{1 V} \vert}^{2} \\ {\vert {g}_{1 H} \vert}^{2} - {\vert {g}_{1 V} \vert}^{2} \\ 2 \vert {g}_{1 H} \vert \vert {g}_{1 V} \vert cos {δ}_{1} \\ 2 \vert {g}_{1 H} \vert \vert {g}_{1 V} \vert sin {δ}_{1}\end{matrix} ⌋ , {S}_{2} = [ \begin{matrix}{s}_{20} \\ {s}_{21} \\ {s}_{22} \\ {s}_{23}\end{matrix} ] = [ \begin{matrix}{\vert {g}_{2 H} \vert}^{2} + {\vert {g}_{2 V} \vert}^{2} \\ {\vert {g}_{2 H} \vert}^{2} - {\vert {g}_{2 V} \vert}^{2} \\ 2 Re [ {g}_{2 H} {g}_{2 V}^{*} ] \\ 2 Im [ {g}_{2 H} {g}_{2 V}^{*} ]\end{matrix} ] = [ \begin{matrix}{\vert {g}_{2 H} \vert}^{2} + {\vert {g}_{2 V} \vert}^{2} \\ {\vert {g}_{2 H} \vert}^{2} - {\vert {g}_{2 V} \vert}^{2} \\ 2 \vert {g}_{2 H} \vert \vert {g}_{2 V} \vert cos {δ}_{2} \\ 2 \vert {g}_{2 H} \vert \vert {g}_{2 V} \vert sin {δ}_{2}\end{matrix} ] & ( 10 )\end{matrix}$$

As shown in formula (10), the zero-th Stokes parameters s₁₀ and s₂₀ indicate the sum of the powers of horizontal components |g_{1H}|² and |g_{2H}|² and the powers of vertical components |g_{1V}|² and |g_{2V}|², that is, the intensity of entire light. The first Stokes parameters indicate the difference between the powers of the horizontal components |g_{1H}|² and |g_{2H}|² and the powers of the vertical components |g_{1V}|² and |g_{2V}|², that is, the difference between the components orthogonal to each other. The second Stokes parameters correspond to twice the real part of the products of complex conjugates of the horizontal and vertical components g_{1H}g_{1V}* and g_{2H}g_{2H}*, that is, the value obtained by multiplying the product of the intensity of each of the horizontal components g_{1H} and g_{2H} and the vertical components g_{1V} and g_{2V}, which are |g_{1H}||g_{1V}| and |g_{2H}||g_{2V}|, and the cosine values cos δ₁ and cos δ₂ of the phase differences δ₁ and δ₂ of the horizontal components and the vertical components by 2. The third Stokes parameters correspond to twice the imaginary part of the products of the complex conjugates of the horizontal and vertical components g_{1H}g_{1V}* and g_{2H}g_{2V}*, that is, the value obtained by multiplying the product of the intensity of each of the horizontal components g_{1H} and g_{2H} and the vertical components g_{1V} and g_{2V}, which are |g_{1H}||g_{1V}| and |g_{2H}||g_{2V}|, and the sine values sin δ₁ and sin δ₂ of the phase differences δ₁ and δ₂ of the horizontal components and the vertical components by 2.

The variance characteristic analysis unit 218 calculates time averages <S₁> and <S₂> in the observation period for each of the first Stokes vector S₁ and the second Stokes vector S₂, and calculates the sum of the zero-th to the third Stokes parameters that are element values as a zero-th time average <s₁₀+s₂₀>, a first time average <s₁₁+s₂₁>, a second time average <s₁₂+s₂₂>, and a third time average <s₁₃+s₂₃> (step S124 in FIG. 18).

Then, the variance characteristic analysis unit 218 determines the value obtained by dividing the square root of the sum of squares of the first time average, the second time average, and the third time average by the zero-th time average as a TPU as shown in formula (11) (step S126 in FIG. 18). The TPU has a smaller value as the temporal variance of the polarization state becomes smaller. Therefore, it is expected that the TPU decreases as a biological tissue of a sample becomes more active. On the contrary, the CPR, the LPR or the temporal variation characteristic value with respect to birefringence have a greater value as the temporal variance of the polarization state becomes significant.

[Math. 11]

TPU=\sqrt{<(s₁₁+s₂₁)²>+<(s₁₂+S₂₂)²>+<(s₁₃+s₂₃)²>}/<|s₁₀+s₂₀|> (11)

Further, the measurement Jones matrix includes a noise component as shown in formula (12). In formula (12), n_{1H}, n_{1V}, n_{2H}, and n_{2V} indicate noise components added to signal components E_{1H}, E_{1V}, E_{2H}, and E_{2V}, respectively.

$$\begin{matrix}[ Math . 12 ] & \\ {J}_{mz} = ⌊ \begin{matrix}{g}_{1 H} & {g}_{2 H} \\ {g}_{1 V} & {g}_{2 V}\end{matrix} ⌋ = ⌊ \begin{matrix}{E}_{1 H} + {n}_{1 H} & {E}_{2 H} + {n}_{2 H} \\ {E}_{1 V} + {n}_{1 V} & {E}_{2 V} + {n}_{2 V}\end{matrix} ⌋ & ( 12 )\end{matrix}$$

The polarization analysis unit 216 can compensate for the noise components by subtracting a time mean power of element values of the noise components from a time mean power of element values included in each of the time average <S₁> of the first Stokes vector and the time average <S> of the second Stokes vector as shown in formula (13). Here, the variance characteristic analysis unit 218 can calculate a TPU from which the noise components have been removed by substituting the time average <S₁> of the first Stokes vector and the time average <S₂> of the second Stokes vector before the correction of the noise components with a time average <S₁′> of the first Stokes vector and the time average <S₂′> of the second Stokes vector for the signal components after the correction of the noise components in formula (11).

$$\begin{matrix}[ Math . 13 ] & \\ < {S}_{1}^{′} >= [ \begin{matrix}< {s}_{10}^{′} > \\ < {s}_{11}^{′} > \\ < {s}_{12}^{′} > \\ < {s}_{13}^{′} >\end{matrix} ] = ⌊ \begin{matrix}< {\vert {g}_{1 H} \vert}^{2} > + < {\vert {g}_{1 V} \vert}^{2} > - ( < {\vert {n}_{1 H} \vert}^{2} > + < {\vert {n}_{1 V} \vert}^{2} > ) \\ < {\vert {g}_{1 H} \vert}^{2} > - < {\vert {g}_{1 V} \vert}^{2} > - ( < {\vert {n}_{1 H} \vert}^{2} > - < {\vert {n}_{1 V} \vert}^{2} > ) \\ 2 \sqrt{< {\vert {g}_{1 H} \vert}^{2} > - < {\vert {n}_{1 H} \vert}^{2} >} \sqrt{< {\vert {g}_{1 V} \vert}^{2} > - < {\vert {n}_{1 V} \vert}^{2} >} cos {δ}_{1} \\ 2 \sqrt{< {\vert {g}_{1 H} \vert}^{2} > - < {\vert {n}_{1 H} \vert}^{2} >} \sqrt{< {\vert {g}_{1 V} \vert}^{2} > - < {\vert {n}_{1 V} \vert}^{2} >} sin {δ}_{1}\end{matrix} ⌋ , \\ < {S}_{2}^{′} >= [ \begin{matrix}< {s}_{20}^{′} > \\ < {s}_{21}^{′} > \\ < {s}_{22}^{′} > \\ < {s}_{23}^{′} >\end{matrix} ] = ⌊ \begin{matrix}< {\vert {g}_{2 H} \vert}^{2} > + < {\vert {g}_{2 V} \vert}^{2} > - ( < {\vert {2}_{2 H} \vert}^{2} > + < {\vert {n}_{2 V} \vert}^{2} > ) \\ < {\vert {g}_{2 H} \vert}^{2} > - < {\vert {g}_{2 V} \vert}^{2} > - ( < {\vert {n}_{2 H} \vert}^{2} > - < {\vert {n}_{2 V} \vert}^{2} > ) \\ 2 \sqrt{< {\vert {g}_{2 H} \vert}^{2} > - < {\vert {n}_{2 H} \vert}^{2} >} \sqrt{< {\vert {g}_{2 V} \vert}^{2} > - < {\vert {n}_{2 V} \vert}^{2} >} cos {δ}_{2} \\ 2 \sqrt{< {\vert {g}_{2 H} \vert}^{2} > - < {\vert {n}_{2 H} \vert}^{2} >} \sqrt{< {\vert {g}_{2 V} \vert}^{2} > - < {\vert {n}_{2 V} \vert}^{2} >} sin {δ}_{2}\end{matrix} ⌋ , & ( 13 )\end{matrix}$$

In addition, the variance characteristic analysis unit 218 may calculate, for example, a von Neumann entropy of a Jones matrix defined for the observation point as a temporal variation characteristic value within the predetermined observation period. The von Neumann entropy is defined by formula (14). In formula (14), λᵢ indicates an eigenvalue of a Hermitian matrix T (described below), and a normalized eigenvalue is obtained by normalizing the individual eigenvalues λᵢ with the sum of the eigenvalues.

$$\begin{matrix}[ Math . 14 ] & \\ H = {\sum }_{i = 1}^{4} - {λ}_{i}^{′} {log}_{4} {λ}_{i}^{′} , {λ}_{i}^{′} = \frac{{λ}_{i}}{{\sum }_{i = 1}^{4} {λ}_{i}} & ( 14 )\end{matrix}$$

$$\begin{matrix}[ Math . 15 ] & \\ E ( R ) = {\sum }_{i = 1}^{4} {λ}_{i}^{′} {R}_{i} & ( 15 )\end{matrix}$$

As shown in formula (15), it is known that an expected value E (R) of the LPR is a weighted mean value of Rᵢ that is the LPR of the optical axis corresponding to the individual eigenvalues. A weight factor multiplied by the LPR of individual optical axes at weighted mean is given to the normalized eigenvalue λᵢ′ corresponding to the optical axis. For this reason, the von Neumann entropy of the Jones matrix defined using the normalized eigenvalue can be regarded as a type of a temporal variation characteristic value for a polarization state. Although the von Neumann entropy of the Jones matrix is described in detail in the following documents, in the present embodiment, randomness caused by a temporal variance of the Jones matrix is evaluated as a polarization characteristic value.

Masahiro Yamanari et. al., “Estimation of Jones Matrix, Birefringence and Entropy using Cloude-Pottier Decomposition in Polarization-Sensitive Optical Coherence Tomography,” Biomedical Optics Express, Vol. 7, No. 9, p. 3551-3572, published Sep. 1, 2016. Masahiro Yamanari et. al., “Estimation of Jones Matrix, Birefringence and Entropy using Cloude-Pottier Decomposition in Polarization-Sensitive Optical Coherence Tomography: Erratum,” Biomedical Optics Express, Vol. 7, No. 11, p. 4636-4637, published Nov. 1, 2016.

The polarization analysis unit 216 and the variance characteristic analysis unit 218 can calculate a von Neumann entropy H in the following procedure. First, the polarization analysis unit 216 configures the four-dimensional vectors [g_{1H} g_{2H} g_{1V} g_{2V}]^{T} configured by arranging elements of two rows and two columns of the measurement Jones matrix J_{mz} for each observation point in order of the rows and columns as a target vector κ_{L}. This target vector κ_{L} indicates substantially the same value as the measurement Jones matrix J_{mz}.

The polarization analysis unit 216 calculates a square matrix κ_{L}κ_{L}⁺ with four rows and four columns by multiplying the target vector κ_{L} by the Hermitian conjugate κ_{L}⁺ of the target vector. The variance characteristic analysis unit 218 determines the time average <κ_{L}κ_{L}⁺> of the square matrix κ_{L}κ_{L}⁺ within a predetermined period as a matrix T as shown in formula (16). The matrix T is a positive semi-definite Hermitian matrix with four rows and four columns.

$$\begin{matrix}[ Math . 16 ] & \\ T = < {κ}_{L} {κ}_{L}^{+} >= ⌊ \begin{matrix}< {\vert {J}_{1 H} \vert}^{2} > & < {J}_{1 H} {J}_{2 H}^{*} > & < {J}_{1 H} {J}_{1 V}^{*} > & < {J}_{1 H} {J}_{2 V}^{*} > \\ < {J}_{1 V} {J}_{1 H}^{*} > & < {\vert {J}_{2 H} \vert}^{2} > & < {J}_{2 H} {J}_{1 V}^{*} > & < {J}_{2 H} {J}_{2 V}^{*} > \\ < {J}_{1 V} {J}_{1 H}^{*} > & < {J}_{1 V} {J}_{2 H}^{*} > & < {\vert {J}_{1 V} \vert}^{2} > & < {J}_{1 V} {J}_{2 V}^{*} > \\ < {J}_{2 V} {J}_{1 H}^{*} > & < {J}_{2 V} {J}_{2 H}^{*} & < {J}_{2 V} {J}_{1 V}^{*} > & < {\vert {J}_{2 V} \vert}^{2} >\end{matrix} ⌋ & ( 16 )\end{matrix}$$

In addition, the variance characteristic analysis unit 218 diagonalizes the matrix T, and calculates four eigenvalues λᵢ (i=1 to 4). However, i is an index determined in descending order of the eigenvalues λᵢ. The variance characteristic analysis unit 218 calculates a normalized eigenvalue λᵢ′ by dividing an individual eigenvalue λᵢ by the sum of the four eigenvalues Σⱼ₌₁⁴λⱼ. The variance characteristic analysis unit 218 can calculate the value obtained by performing positive/negative reversal on the sum of the product of the normalized eigenvalue λᵢ′ and the logarithmic value log (λᵢ′) thereof as a von Neumann entropy H as shown in formula (14). The von Neumann entropy has a value of 0 or more and 1 or less. The von Neumann entropy H has the value 1 for a set of a fully random Jones matrices. However, the base of the logarithmic value in formula (14) is 4.

Further, the variance characteristic analysis unit 218 may determine a von Neumann entropy Hₛ of a signal component by subtracting a von Neumann entropy Hₙ of a noise component from a von Neumann entropy Hₘ determined based on the measurement Jones matrix as shown in formula (17).

[Math. 17]

Hₛ=Hₘ−Hₙ (17)

The variance characteristic analysis unit 218 can cause the von Neumann entropy Hₙ (E₁, E₂) of the noise component to approximate to the sum of a first noise component entropy H (E₁) that is an entropy of a first noise component n₁ and a second noise component entropy H (E₂) that is an entropy of a second noise component n₂ each added to a first Jones vector E₁ and a second Jones vector E₂ constituting a partial space of the Jones matrix J_{mz} as shown in formula (18). However, the first noise component n₁ and the second noise component n₂ are assumed to be independent of each other.

[Math. 18]

Hₙ(E₁,E₂)=Hₙ(E₁)+Hₙ(E₂) (18)

The variance characteristic analysis unit 218 can perform positive/negative reversal on the total of a j-th eigenvalue ζⱼ⁽ⁱ⁾ and a logarithmic value log(ζⱼ⁽ⁱ⁾) of an i-th noise component to calculate an i-th noise component entropy H(Eᵢ) as shown in formula (19).

$$\begin{matrix}[ Math . 19 ] & \\ {H}_{n} ( {E}_{i} ) = {\sum }_{j = 1}^{2} - {ζ}_{j}^{( i )} log ( {ζ}_{j}^{( i )} ) & ( 19 )\end{matrix}$$

However, the variance characteristic analysis unit 218 can calculate the value obtained by dividing the value obtained by adding P(i) that is a TPU of an i-th Jones vector to 1 or dividing P(i) by 1 by 2 as a first eigenvalue ζ₁⁽ⁱ⁾ and a second eigenvalue ζ₂⁽ⁱ⁾ as shown in formula (20).

$$\begin{matrix}[ Math . 20 ] & \\ {ζ}_{j}^{( i )} = \frac{1 \pm {P}^{( i )}}{2} & ( 20 )\end{matrix}$$

The variance characteristic analysis unit 218 can calculate P⁽ⁱ⁾ that is a TPU of the i-th Jones vector by dividing the square root of the sum of squares of the time averages of the first to the third Stokes parameters of the first Stokes vector and the second Stokes vector after the correction of the noise components by the time average of a zero-th Stokes parameter as shown in formula (21).

[Math. 21]

P⁽ⁱ⁾=\sqrt{<|sᵢ₁′|>+<|sᵢ₂′|²>+<|sᵢ₃′|²>}/<|sᵢ₀|> (21)

Calculation Example of Temporal Variation Characteristic Value

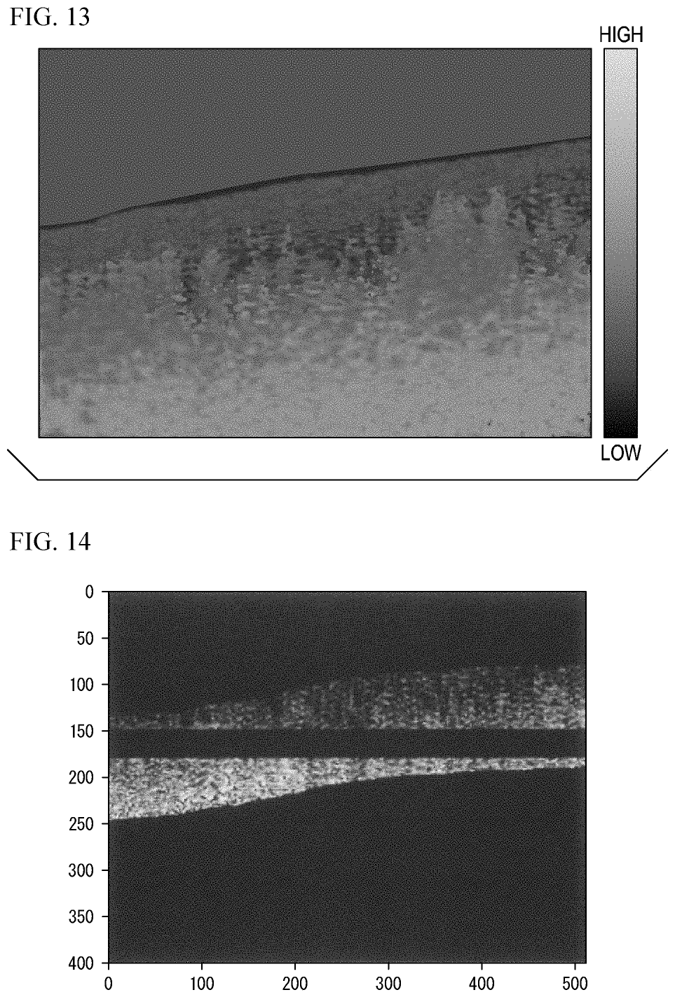

Next, an example of calculating the above-mentioned temporal variation characteristic value will be described. FIG. 13 is a diagram illustrating an example of a spatial distribution of variance of birefringences. In FIG. 13, the variance of the birefringences per observation point in a biological tissue is indicated in shades. Observation points are distributed in the x-z plane. Birefringences are more largely dispersed in brighter portions, and birefringences are narrowly dispersed in darker portions. In FIG. 13, the black-filled upper portion indicates the outside of the tissue. There is a tendency that the birefringences are more largely dispersed in the inside of the tissue than on the surface as a whole.

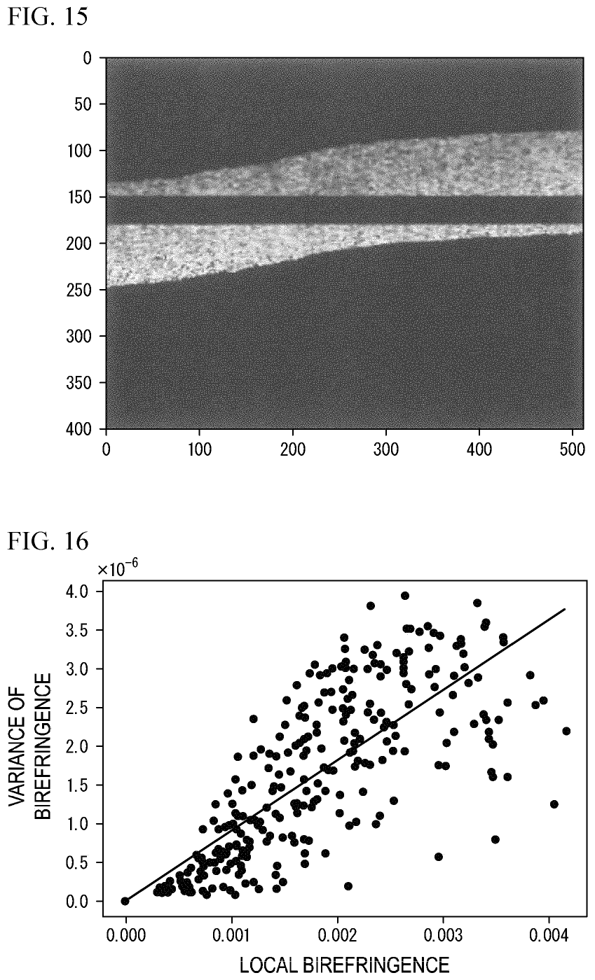

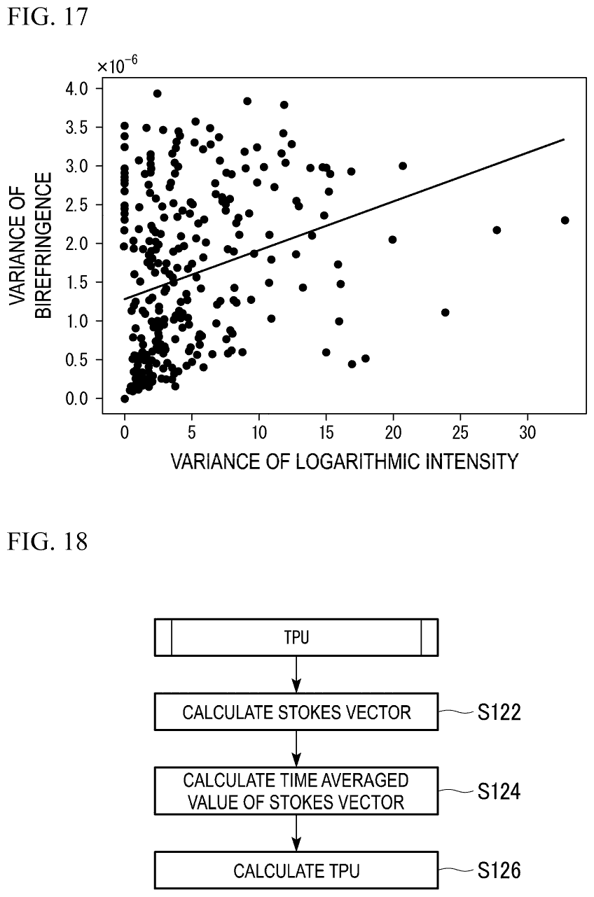

FIG. 14 is a diagram illustrating another example of a spatial distribution of the variance of birefringences. In FIG. 14, the variance of the birefringences per observation point in a biological tissue is indicated in shades. FIG. 15 illustrates the mean local birefringence for each observation point. In the example illustrated in FIG. 15, the same biological tissue as in FIG. 14 was observed. However, in the example shown in FIG. 14, a biological tissue different from that in FIG. 13 was observed. In FIG. 14 and FIG. 15, the portions brighter than the surroundings indicate the distribution range of the biological tissue. In both drawings, there is a tendency that the birefringence or the mean local birefringence are relatively high in the lower part of the drawings, and the birefringence or the mean local birefringence are relatively high in the left part of the drawings. FIG. 16 is a diagram illustrating an example of a correlation between the variance of the birefringences and the mean local birefringences. In FIG. 16, the vertical axis represents the variance of the birefringences, and the horizontal axis represents the mean local birefringences. FIG. 16 indicates that there is a significant correlation between the local birefringences and the variance of the birefringences. The correlation coefficient was 0.776. This proves that there is a tendency that birefringences are largely dispersed as a local birefringence becomes higher. FIG. 17 is a diagram illustrating an example of a correlation between the variance of birefringences and the variance of logarithmic intensities. In FIG. 17, the vertical axis represents the variance of the birefringences, and the horizontal axis represents the variance of logarithmic intensities. Each logarithmic intensity is the logarithmic value of a signal intensity per observation point. FIG. 17 indicates that there is no significant correlation between the local birefringences and the variance of the birefringences. The correlation coefficient was 0.280. This proves that a logarithmic intensity alone does not represent a polarization state.

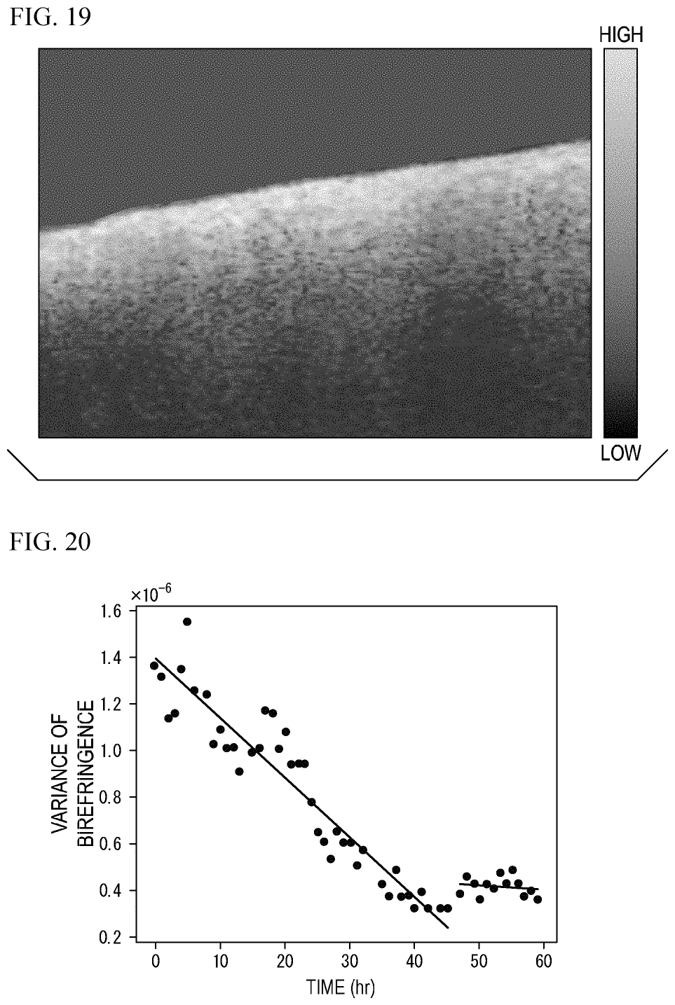

FIG. 19 is a diagram illustrating an example of a spatial distribution of TPUs. In FIG. 19, a TPU per observation point in a biological tissue is indicated in shades. In the example illustrated in FIG. 19, the same biological tissue as in FIG. 13 was observed. A bright portion indicates a greater TPU, and a darker portion indicates a smaller TPU. There is a tendency that a TPU becomes smaller in the inside of the tissue than on the surface as a whole. This tendency indicates the opposite tendency to the variance of birefringences. While a temporal change in a polarization state becomes smaller as a TPU becomes greater, a temporal change in a polarization state becomes greater as the birefringences are more largely dispersed.

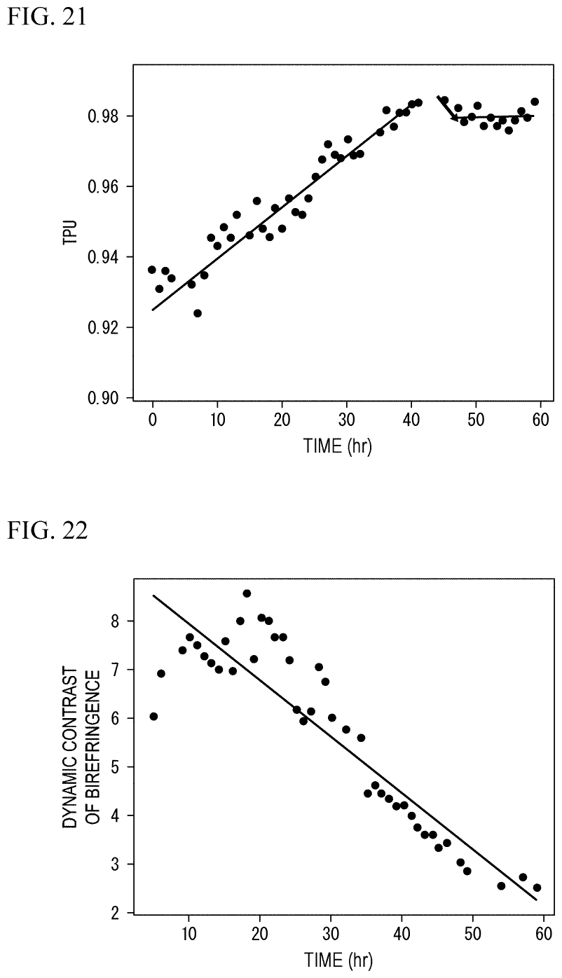

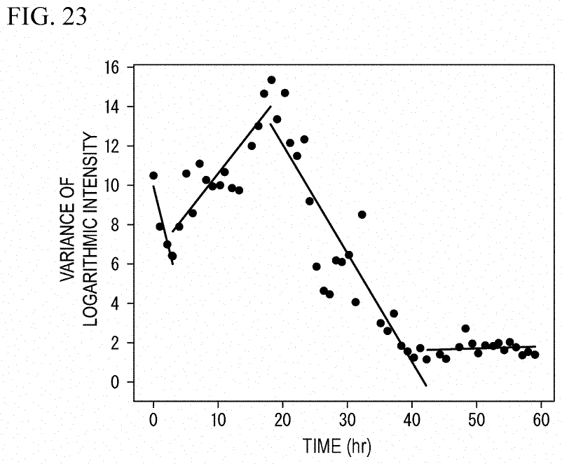

FIG. 20 is a diagram illustrating an example of a temporal change in the variance of birefringences. FIG. 21 is a diagram illustrating another example of a temporal change in TPUs. FIG. 22 is a diagram illustrating an example of a temporal change in dynamic contrast of birefringences. FIG. 23 is a diagram illustrating an example of a temporal change in the variance of logarithmic intensities. FIG. 20, FIG. 21, FIG. 22, and FIG. 23 illustrate the variance of birefringences, TPUs, birefringences, and the variance of logarithmic intensities on an hourly basis at a certain observation point within a common biological tissue, respectively. The biological tissue has a decreasing active state over time.

In FIG. 20, while the tendency of the variance of birefringences significantly decreasing over time until 44 hours elapsed from the time 0 is shown, the birefringence was substantially constant after the elapse of 45 hours until 60 hours elapsed. FIG. 20 shows the tendency of the variance of birefringences significantly decreasing over time until 44 hours elapsed from the time 0. The correlation coefficient was −0.9486. Meanwhile, the birefringence was substantially constant after the elapse of 45 hours until 60 hours elapsed. The correlation coefficient was −0.1711. FIG. 21 shows the tendency of TPUs significantly increasing over time until 42 hours elapsed from the time 0. The correlation coefficient was 0.9413. On the other hand, the TPU was substantially constant from the elapse of 43 hours until 60 hours elapsed. The correlation coefficient was 0.0735. FIG. 22 shows a tendency of dynamic contrast of birefringences significantly decreasing over time. The correlation coefficient was −0.905. In FIG. 20, the variance of the logarithmic intensity decreased from the time 0 until three hours elapsed, the variance of the logarithmic intensity increased after the elapse of four hours until 19 hours elapsed, the variance of the logarithmic intensity decreased after the elapse of 20 hours until 42 hours elapsed, and the variance of the logarithmic intensity was substantially constant after the elapse of 42 hours until 60 hours elapsed. According to these examples of the present embodiment, a temporal variation characteristic value of a polarization characteristic value for each observation point in a sample can be measured, and an active state of a biological tissue can be evaluated based on the measured temporal variation characteristic value.

As described above, the measurement signal processing device 200 according to the above-described embodiment includes the polarization analysis unit 216 that determines a polarization characteristic value based on polarization characteristics at an observation point in a sample, based on a first measurement signal (e.g., the first horizontally polarized spectral interferometric signal) of a first interferometric component in a first polarization state (e.g., horizontal polarization), the first interferometric component obtained by causing a first incidence component (e.g., a horizontal polarization component) incident on a sample in the first polarization state to interfere with a component obtained by reflection or scattering of the first incidence component from the sample, a second measurement signal (e.g., the second horizontally polarized spectral interferometric signal) in a second polarization state (e.g., vertical polarization) with respect to the first interferometric component, a third measurement signal (e.g., the first vertically polarized spectral interferometric signal) of the first interferometric component in the first polarization state, the first interferometric component being obtained by causing the first incidence component incident on a sample in the first polarization state to interfere with a component obtained by reflection or scattering of a second incidence component (e.g., a vertical polarization component) incident on the sample in a second polarization state from the sample, and a fourth measurement signal (e.g., the second vertically polarized spectral interferometric signal) in the second polarization state with respect to the second interferometric component, and the variance characteristic analysis unit 218 that determines a temporal variation characteristic value indicating a temporal variation characteristic of the polarization characteristic value. With this configuration, a temporal variation characteristic value of a polarization characteristic value for each observation point in the sample is determined. Because the temporal variance of the polarization characteristic value is significantly correlated with an activity of a tissue, the activity of the tissue can be quantitatively evaluated using a distribution of temporal variation characteristic values determined for each of observation points.

Further, the polarization analysis unit 216 may determine a Jones matrix for each observation point based on the first measurement signal, the second measurement signal, the third measurement signal, and the fourth measurement signal, determine a cumulative Jones matrix for an observation point from a Jones matrix for the observation point in the sample and a Jones matrix for a surface of the sample, and determine a CPR that is a phase difference between the eigenvalues of the cumulative Jones matrix as a polarization characteristic value. With this configuration, the temporal variation characteristic value of the CPR corresponding to the phase difference between the polarization components of the surface of the sample and the observation point can be utilized in evaluating the activity of the tissue.

Further, the polarization analysis unit 216 may determine a Jones matrix for each observation point based on the first measurement signal, the second measurement signal, the third measurement signal, and the fourth measurement signal, determine a local Jones matrix for a first observation point and a second observation point from a Jones matrix for the first observation point in the sample and a Jones matrix for the second observation point in the sample, and determine an LPR that is a phase difference between two eigenvalues of the local Jones matrix. With this configuration, the temporal variation characteristic value of the LPR corresponding to the phase difference between the polarization components generated in the section of the first observation point and the second observation point can be utilized in evaluating the activity of the tissue. As a result, the activity of the tissue can be evaluated for each minute region.

Furthermore, the polarization analysis unit 216 may determine a birefringence by dividing a local phase retardation by the wavenumber of incident light incident on the sample and the thickness of the first observation point and the second observation point. Thus, it is possible to evaluate the activity of the tissue for each minute region, and the birefringence may be used to facilitate the comparison of birefringences of other tissues or existing substances to be observed.

The variance characteristic analysis unit 218 may calculate a temporal variation characteristic value based on the variance or standard deviation of the polarization characteristic value. Thus, the magnitude of the temporal variance of the polarization characteristic value can be quantitatively evaluated.

The variance characteristic analysis unit 218 may calculate a temporal variation characteristic value based on the variance or standard deviation of the logarithmic value of the polarization characteristic value. In the course of calculating the variance or standard deviation, the constant potentially multiplied by the polarization characteristic value is erased, and thus the substantial temporal variance of the polarization characteristic value can be evaluated. In addition, by taking the logarithmic value, the comparison of temporal variation characteristic values of the other tissues and existing substances to be observed having different scales becomes easier.

The variance characteristic analysis unit 218 may calculate the dynamic contrast by dividing the standard deviation of the polarization characteristic value by the mean value of birefringences. By dividing by the mean value of the polarization characteristic values, the standard deviation of the polarization characteristic values is normalized, and thus the substantial temporal variance of the polarization characteristic values can be evaluated without changing the scale.

The polarization analysis unit 216 may convert a first Jones vector based on the first measurement signal and the second measurement signal and a second Jones vector based on the third measurement signal and the fourth measurement signal into a first Stokes vector and a second Stokes vector, respectively, as polarization characteristic values, and the variance characteristic analysis unit 218 may determine a TPU based on the time average of the first Stokes vectors and the time average of the second Stokes vectors as a temporal variation characteristic value. According to this configuration, the uniformity of the polarization states at observation points according to the elapse of time can be quantified by using the TPU. The TPU tends to be greater as the activity of a tissue becomes lower. Therefore, an inactive state of a tissue can be quantitatively evaluated using a distribution of TPUs determined for each of observation points.

The variance characteristic analysis unit 218 may determine a TPU based on the time average of the first Stokes vectors after correction of subtracting a noise component from the first Stokes vectors and the time average of the second Stokes vectors after correction of subtracting a noise component from the second Stokes vectors. According to this configuration, the noise components are removed from the first Stokes vector and the second Stokes vector, and the signal component is left. Thus, the influence of noise on the TPU can be suppressed, and the activity of the tissue can be accurately evaluated.

The polarization analysis unit 216 may determine a Jones matrix for each observation point from the first measurement signal, the second measurement signal, the third measurement signal, and the fourth measurement signal as the polarization characteristic value, and the variance characteristic analysis unit 218 may calculate a von Neumann entropy of the Jones matrix as the temporal variation characteristic value. According to this configuration, the randomness of the Jones matrix indicating the polarization state at the observation point can be quantified. Therefore, the activity of the tissue can be quantitatively evaluated using a distribution of the von Neumann entropy determined for each of observation points.

The variance characteristic analysis unit 218 may calculate an entropy of a noise component from a temporal polarization uniformity of the first Stokes vector and a temporal polarization uniformity of the second Stokes vector converted from the first Jones vector based on the first measurement signal and the second measurement signal and the second Jones vector based on the third measurement signal and the fourth measurement signal, respectively, and correct the von Neumann entropy based on the entropy of the noise component. According to this configuration, the contribution of the entropy of the noise component is compensated for by the von Neumann entropy, and the von Neumann entropy of the signal component is obtained. Thus, the influence of noise on the von Neumann entropy can be suppressed, and the activity of the tissue can be accurately evaluated.

The measurement signal processing device 200 may include the image processing unit 220 configured to generate image data having, as a signal value, an output value of a temporal variation characteristic value for each observation point using a function to provide an output value monotonically changing with respect to a change in an input value. According to this configuration, an image having a distribution of luminance or tone corresponding to the temporal variation characteristic value for each observation point is obtained. The measurement signal processing device 200 may include the output processing unit 222 that determines an evaluation value indicating an active state of a biological tissue provided as a sample based on a temporal variation characteristic value. Such an evaluation value may be, for example, a real value that increases as the degree of activity becomes higher. For example, the output processing unit 222 sets a function indicating the relationship between an evaluation value and the temporal variation characteristic value and parameters thereof in advance. The output processing unit 222 may store the evaluation values in the storage unit 230, or may output the values to other devices. The image processing unit 220 may convert the evaluation value calculated for each observation point by the output processing unit 222 into a signal value for each pixel as described above, and generate image data having the converted signal value. As a result, a user can easily evaluate the activity of the tissue in an observation region when he or she sees the obtained image.

Although the embodiments of the present invention have been described above in detail with reference to the drawings, specific configurations are not limited to those described above, and various changes in design or the like may be made within the scope that does not depart from the gist of the present invention.

For example, although the example in which the evaluation device 20 and the measurement signal processing device 200 are a part of the OCT system 1, respectively, in the above description, the invention is not limited thereto. The evaluation device 20 and the measurement signal processing device 200 may be independent from the OCT system 1 and may be a single device not including an optical system. In this case, the optical system control unit 212 may be omitted in the control unit 210 of the measurement signal processing device 200. Likewise, a control means for controlling the optical system may be omitted in the evaluation device 20. The evaluation device 20 and the measurement signal acquisition unit 214 are not limited to the optical system, and may acquire detection signals and measurement signals from other devices such as a data storage device and a PC wirelessly or by wire, for example, via a network. Further, the measurement signal processing device 200 may include any of the input/output unit 240, the display unit 250, and the operation unit 260 as described above, and some or all of them may be omitted. Further, the evaluation device 20 may include any of functional configurations corresponding to the input/output unit 240, the display unit 250, and the operation unit 260, and some or all of them may be omitted. Further, one or both of the image processing unit 220 and the output processing unit 222 may be omitted in the control unit 210 of the measurement signal processing device 200. In a case where the image processing unit 220 is omitted, the control unit 210 may output data indicating a generated temporal variation characteristic value to other devices, such as data storage devices, PCs, or other image processing devices wirelessly or by wire, and may output the data, for example, via a network. Further, the evaluation device 20 may include any of functional configurations corresponding to one or both of the image processing unit 220 and the output processing unit 222, and some or all of them may be omitted. A device serving as an output destination may have a function similar to that of the image processing unit 220, that is, a function of generating the output image data based on the data input from the evaluation device 20 or the measurement signal processing device 200 and displaying an image based on the generated output image data. In the evaluation device 20, a functional configuration corresponding to one or both of the image processing unit 220 and the output processing unit 222 of the measurement signal processing device 200 may be omitted. In addition, a part or all of the evaluation device 20 or the measurement signal processing device 200 according to the embodiments described above may be implemented as an integrated circuit such as a large scale integration (LSI). Each functional block of the evaluation device 20 or the measurement signal processing device 200 may be individually implemented as a processor, or a part or all thereof may be integrated and implemented as a processor. In addition, the method for implementation as an integrated circuit is not limited to LSI, and implementation may be achieved with a dedicated circuit or a general-purpose processor. In addition, if a technology for implementation as an integrated circuit to replace LSI is developed as a result of improvement of semiconductor technology, an integrated circuit according to the technology may be used. Although the preferred embodiments of the present invention have been described above, the present invention is not limited to these embodiments and modified examples thereof. In the scope that does not depart from the spirit of the present invention, additions, omission, substitutions, and other changes of the configuration can be made. Furthermore, the present invention is not limited by the foregoing description, and is limited only by the appended claims.

### INDUSTRIAL APPLICABILITY

According to the embodiments described above, for example, the invention is highly useful in regenerative medical culture tissue, organoid quality control, animal experiments, pharmaceutical evaluation for measurement of efficacy using cultured tissues, treatment effect evaluation in gene therapy of fundus photoreceptor cells, and the like.

### REFERENCE SIGNS LIST

1 OCT system
10 Imager
20 Evaluation device
22 Measurer
24 Evaluator
102 Light source
110 Polarization delay unit
128 Probe
130 Reference arm
150 Polarization diversity detection unit
200 Measurement signal processing device
210 Control unit
212 Optical system control unit
214 Measurement signal acquisition unit
216 Polarization analysis unit
218 Variance characteristic analysis unit
220 Image processing unit
222 Output processing unit
230 Storage unit
240 Input/output unit
250 Display unit
260 Operation unit

1. An evaluation device comprising:
a measurement circuitry configured to acquire an optical coherence tomography (OCT) signal indicating a state of a biological tissue provided as a sample and to acquire a signal value based on the OCT signal at an observation point in the sample; and
an evaluation circuitry configured to calculate a temporal variation characteristic value indicating a temporal variation characteristic of the signal value within a predetermined period.

2. The evaluation device according to claim 1, wherein
the evaluation circuitry calculates a variance of the signal value as the temporal variation characteristic value.

3. The evaluation device according to claim 2, wherein
the evaluation circuitry divides a sum of squares of a deviation between a signal intensity of the OCT signal and a mean value of the signal intensity at a frame time within the predetermined period by the number of frames in the predetermined period to calculate the variance at the observation point.

4. The evaluation device according to claim 1, wherein
the evaluation circuitry calculates a correlation coefficient of the signal value and a time-shifted signal value obtained by time-shifting the signal value by a time shift amount τ for each time shift amount τ, and calculates a decay speed of the correlation coefficient according to an increase in the time shift amount τ as the temporal variation characteristic value.

5. The evaluation device according to claim 4, wherein
the evaluation circuitry
calculates, as a variance, a sum of squares of a deviation between a signal intensity of the OCT signal and a mean value of the signal intensity at a frame time within the predetermined period,
calculates, as a covariance, a sum of a product of a deviation between a signal intensity of the OCT signal and a mean value of the signal intensity at a frame time within the predetermined period and another deviation between a time-shifted signal intensity of the OCT signal at a shift time shifted from the frame time by a time shift amount τ and a mean value of the time-shifted signal intensity,
calculates the correlation coefficient by dividing the covariance by the variance for each shift amount τ, and
performs regression analysis using a predetermined decay function using the correlation coefficient for each time shift amount τ and calculates a parameter of the decay function approximating the correlation coefficient, as the decay speed at an observation point.

6. The evaluation device according to claim 4, wherein
the evaluation circuitry calculates the decay speed using the correlation coefficient calculated with the time shift amount τ being non-zero.

7. The evaluation device according to claim 1, wherein
the measurement circuitry determines a polarization characteristic value based on a polarization characteristic at an observation point in the sample, based on a first measurement signal of a first interferometric component in a first polarization state, the first interferometric component being obtained by causing a first incidence component incident on the sample in the first polarization state to interfere with a component obtained by reflection or scattering of the first incidence component from the sample, a second measurement signal in a second polarization state with respect to the first interferometric component, a third measurement signal of a second interferometric component in the first polarization state, the second interferometric component being obtained by causing a second incidence component incident on the sample in the second polarization state to interfere with a component obtained by reflection or scattering of the second incidence component from the sample, and a fourth measurement signal in the second polarization state with respect to the second interferometric component, and
the evaluation circuitry determines the temporal variation characteristic value indicating a temporal variation characteristic of the polarization characteristic value.

8. The evaluation device according to claim 7, wherein
the measurement circuitry
determines a Jones matrix at an observation point based on the first measurement signal, the second measurement signal, the third measurement signal, and the fourth measurement signal, and determines a cumulative Jones matrix at the observation point from a Jones matrix at the observation point in the sample and a Jones matrix on a surface of the sample, and
determines, as the polarization characteristic value, a cumulative phase retardation index value that is a phase difference between eigenvalues of the cumulative Jones matrix.

9. The evaluation device according to claim 7, wherein
the measurement circuitry determines a Jones matrix at an observation point based on the first measurement signal, the second measurement signal, the third measurement signal, and the fourth measurement signal, and determines, from a Jones matrix at a first observation point in the sample and a Jones matrix at a second observation point in the sample, a local Jones matrix between the first observation point and the second observation point, and
determines the polarization characteristic value based on a local phase retardation that is a phase difference between eigenvalues of the local Jones matrix.

10. The evaluation device according to claim 9, wherein
the measurement circuitry determines a birefringence by dividing the local phase retardation by a wavenumber of incident light incident on the sample and a thickness between the first observation point and the second observation point.

11. The evaluation device according to claim 10, wherein
the evaluation circuitry calculates the temporal variation characteristic value based on a variance or a standard deviation of the polarization characteristic value.

12. The evaluation device according to claim 11, wherein
the evaluation circuitry calculates the temporal variation characteristic value based on a variance or a standard deviation of a logarithmic value of the polarization characteristic value.

13. The evaluation device according to claim 11, wherein
the evaluation circuitry calculates a dynamic contrast by dividing the standard deviation of the polarization characteristic value by a mean value of the birefringence.

14. The evaluation device according to claim 7, wherein
the measurement circuitry converts, as the polarization characteristic values, a first Jones vector based on the first measurement signal and the second measurement signal and a second Jones vector based on the third measurement signal and the fourth measurement signal into a first Stokes vector and a second Stokes vector, respectively, and
the evaluation circuitry determines a temporal polarization uniformity based on a time average of the first Stokes vectors and a time average of the second Stokes vectors as the temporal variation characteristic value.

15. The evaluation device according to claim 14, wherein
the measurement circuitry determines a temporal polarization uniformity based on a time average of a corrected first Stokes vector obtained by subtracting a noise component from the first Stokes vector and a time average of a corrected second Stokes vector obtained by subtracting a noise component from the second Stokes vector.

16. The evaluation device according to claim 7, wherein
the measurement circuitry determines, as the polarization characteristic value, a Jones matrix at an observation point based on the first measurement signal, the second measurement signal, the third measurement signal, and the fourth measurement signal, and
the evaluation unit calculates a von Neumann entropy of the Jones matrix as the temporal variation characteristic value.

17. The evaluation device according to claim 16, wherein
the evaluation circuitry calculates an entropy of a noise component from a temporal polarization uniformity of a first Stokes vector and a temporal polarization uniformity of a second Stokes vector, the first Stokes vector and the second Stokes vector being obtained by conversion from a first Jones vector based on the first measurement signal and the second measurement signal and a second Jones vector based on the third measurement signal and the fourth measurement signal, respectively, and corrects the von Neumann entropy based on the entropy of the noise component.

18. The evaluation device according to claim 7, wherein
the first polarization state is horizontal polarization, and the second polarization state is vertical polarization,
the first measurement signal is a first horizontally polarized spectral interferometric signal,
the second measurement signal is a second horizontally polarized spectral interferometric signal,
the third measurement signal is a first vertically polarized spectral interferometric signal, and
the fourth measurement signal is a second vertically polarized spectral interferometric signal.

19. The evaluation device according to claim 1, wherein
the evaluation circuitry calculates the temporal variation characteristic value on a per observation period interval basis, the observation period interval being longer than the predetermined period.

20. The evaluation device according to claim 1, further comprising:
an output processing circuitry configured to determine an evaluation value indicating an active state of the sample based on the temporal variation characteristic value.

21. The evaluation device according to claim 1, further comprising:
an image processing circuitry configured to generate image data having, as a signal value, an output value for the temporal variation characteristic value at the observation point using a function to provide the output value monotonically changing with respect to a change in an input value.

22. An evaluation method for an evaluation device comprising:
acquiring an optical coherence tomography (OCT) signal indicating a state of a biological tissue provided as a sample and acquiring a signal value based on the OCT signal at an observation point in the sample; and
calculating a temporal variation characteristic value indicating a temporal variation characteristic of the signal value within a predetermined period.

23. A non-transitory computer readable medium storing instructions executable by a processor, wherein execution of the instructions causes the processor to perform:
a measurement procedure of
acquiring an optical coherence tomography (OCT) signal indicating a state of a biological tissue provided as a sample, and
acquiring a signal value based on the OCT signal at an observation point in the sample; and
an evaluation procedure of calculating a temporal variation characteristic value indicating a temporal variation characteristic of the signal value within a predetermined period.
